(19) United States
(12) Patent Application Publication
(10) Pub. No.: US 2025/0378906 A1
(43) Pub. Date: Dec. 11, 2025

(54) METHODS AND ARRANGEMENTS FOR INTERACTIVE SIMULATION OF XRNA PRODUCTION

(71) Applicant: Arranta Bio Holdings, LLC, Watertown, MA (US)

(72) Inventors: Edita Botonjic-Sehic, Watertown, MA (US); Syed Kaschif Ahmed, Watertown, MA (US); Hossein Hamedi, Watertown, MA (US); Aaron Blake Cowley, Watertown, MA (US); Busuyi Adebayo, Watertown, MA (US); Jing Zhu, Watertown, MA (US)

(21) Appl. No.: 19/230,524

(22) Filed: Jun. 6, 2025

Related U.S. Application Data
(60) Provisional application No. 63/657,198, filed on Jun. 7, 2024.

(51) Int. Cl. G16B 20/00; G16B 40/20

(52) U.S. Cl. CPC G16B 20/00; G16B 40/20

(57) ABSTRACT

Logic may interact, via one or more interface models, with a client to obtain client input to determine client data for a sequence for a type of ribonucleic acid (RNA). Logic may analyze, via one or more intuitive models, the client data based on historical batch data and experimental batch data to identify additional client data to achieve one or more target metrics of the client data. And logic may simulate, by one or more process models, continuous RNA production based on the client data and the additional client data to generate one or more experimental outcomes of the continuous RNA production defined by the client data and to output a development plan for the continuous RNA production. And logic may amend the client data and the additional client data after each iteration of simulation and perform additional iterations of the simulation until one or more target metrics are met.

### CROSS REFERENCE TO RELATED APPLICATIONS

This application claims priority under 35 USC § 119 from U.S. Provisional Application No. 63/657,198, entitled “METHODS AND ARRANGEMENTS FOR INTERACTIVE SIMULATION OF xRNA PRODUCTION”, filed on Jun. 7, 2024, the subject matter of which is incorporated herein by reference.

### BACKGROUND

Most biopharmaceuticals are manufactured using batch production methods in which human intervention is required to process a set quantity of material to be produced at the same time. Batch operations may require as long as 1-2 months or more from bioreactor to final formulated product. An alternative approach is continuous manufacturing which is attractive due to its potential to reduce costs while increasing productivity and improving product consistency. Continuous manufacturing processes have been developed in the chemical, petrochemical, food, and mechanical industries. In these contexts, continuous processes have demonstrated less reliance on human labor and fewer gaps in transitioning between unit operations in the process resulting in increased productivity, while the smaller facility footprint required by a continuous process reduces facility costs.

There is a need for continuous manufacturing systems in the biopharmaceutical sector, as an alternative to the more time consuming, resource intensive, and expensive batch processes that represent the current standard of practice, as acknowledged by regulatory agencies which have urged the adoption of continuous biomanufacturing in this sector. See National Academies of Sciences, Engineering and Medicine. Continuous manufacturing for the modernization of pharmaceutical production. 2019.

While real-world experimentation remains vital for certain situations, relying solely on it for process development and testing can be inefficient, costly, and even dangerous. Simulators, on the other hand, offer a compelling alternative, enabling safe, controlled, and iterative exploration of various scenarios. Online simulators can enable wider adoption of the technology.

### BRIEF SUMMARY

Embodiments may include various types of subject matter such as methods, apparatuses, systems, storage media, and/or the like. One embodiment may include a system comprising: memory; and logic circuitry coupled with the memory. In some embodiments, the logic circuitry may interact, via one or more interface models, with a client to obtain client input to determine client data for a sequence for a type of ribonucleic acid (RNA). The logic circuitry may analyze, via the one or more intuitive models, the client data based on historical batch data and experimental batch data to identify additional client data to achieve one or more target metrics of the client data. The logic circuitry may also simulate, by one or more process models, continuous RNA production based on the client data and the additional client data to generate one or more experimental outcomes of the continuous RNA production defined by the client data and to output a development plan for the continuous RNA production.

Another embodiment may comprise a non-transitory storage medium containing instructions, which when executed by a processor, cause the processor to perform operations. The operations may interact, via one or more interface models, with a client to obtain client input to determine client data for a sequence for a type of ribonucleic acid (RNA). The operations may analyze, via the one or more intuitive models, the client data based on historical batch data and experimental batch data to identify additional client data to achieve one or more target metrics of the client data. The operations may simulate, by one or more process models, continuous RNA production based on the client data and the additional client data to generate one or more experimental outcomes of the continuous RNA production defined by the client data and to output a development plan for the continuous RNA production.

Yet another embodiment may comprise a system. The system may comprise data storage and one or more servers coupled with the data storage. The logic circuitry may interact, via one or more interface models of the model library, with a client to obtain client input to determine client data for a sequence for a type of ribonucleic acid (RNA). The logic circuitry may also analyze, via one or more intuitive models of the model library, the client data based on historical batch data of the historical dataset and experimental batch data of the literature dataset to identify additional client data to achieve one or more target metrics of the client data. The logic circuitry may store the client data and the additional data in a client dataset of the data storage. The logic circuitry may simulate, by one or more process models of the model library, continuous RNA production based on the client data and the additional client data to generate one or more experimental outcomes of the continuous RNA production defined by the client data and to output a development plan for the continuous RNA production.

### BRIEF DESCRIPTION OF THE SEVERAL VIEWS OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. In the figures, identical or nearly identical or equivalent elements are typically represented by the same reference characters, and similar elements are typically designated with similar reference numbers, with redundant description omitted. For purposes of clarity and simplicity, not every element is labeled in every figure, nor is every element of each embodiment shown where illustration is not necessary to allow those of ordinary skill in the art to understand the disclosure.

To easily identify the discussion of any particular element or act, the most significant digit or digits in a reference number refer to the figure number in which that element is first introduced.

FIG. 1 illustrates an aspect of hierarchical ingestion of manufacturing data into process simulation platforms, in accordance with one embodiment.

FIG. 2A depicts an embodiment of a system to simulate continuous RNA production through interaction with a client;

FIG. 2B depicts further embodiments of systems including servers, networks, data servers, and software applications to simulate continuous RNA production such as the system shown in FIG. 2A;

FIG. 2C depicts further embodiments of systems including servers, networks, data servers, and software applications to simulate continuous RNA production such as the system shown in FIG. 2A;

FIG. 2D depicts an embodiment of a neural network of a model, such as the models in illustrated in FIG. 2B and FIG. 2C;

FIG. 3A depicts an embodiment of a graphical user interface (GUI) for client interaction with simulator logic circuitry, such as the simulator logic circuitry in illustrated in FIG. 2B to FIG. 2C;

FIG. 3B illustrates an aspect of a GUI for integration of vendor-specific designs across all unit operations in the process simulation platform, in accordance with one embodiment.

FIG. 3C illustrates an aspect of a GUI for a Model Simple Simulator (desktop or installable version) for evaluating mRNA yield and nucleotide utilization, in accordance with one embodiment.

FIG. 3D illustrates another aspect of a GUI for a Model Simple Simulator (web site version) for evaluating mRNA yield and nucleotide utilization, in accordance with one embodiment.

FIG. 3E illustrates an aspect of a GUI for an IVT Model Advanced Simulator (desktop/installable version) for mRNA Synthesis and Nucleotide Conversion Analysis in accordance with one embodiment.

FIG. 3F illustrates an aspect of a GUI for an IVT Model Advanced Simulator (web site version) for mRNA Synthesis and Nucleotide Conversion Analysis in accordance with one embodiment.

FIG. 3G illustrates an aspect of a GUI for a Multi-Objective Optimization Interface (desktop or installable version) for In Vitro Transcription (IVT) Process, in accordance with one embodiment.

FIG. 3H illustrates another aspect of a GUI for a Multi-Objective Optimization Interface (web site version) for In Vitro Transcription (IVT) Process, in accordance with one embodiment.

FIG. 4A depicts an embodiment of a flow diagram for simulator logic circuitry, such as the simulator logic circuitry shown in FIG. 2B and FIG. 2C;

FIG. 4B illustrates an aspect of plots for a GUI having different unit operation process parameters simulated, in accordance with one embodiment.

FIG. 5 depicts a flowchart of embodiments of processes to simulate continuous xRNA production to generate a development plan and scale-up the development plan from pilot development to a full-scale production plan, by simulator logic circuitry, such as the simulator logic circuitry shown in FIG. 2B to FIG. 2C and FIG. 4A;

FIG. 6 depicts an embodiment of a system including a multiple-processor platform, a chipset, buses, and accessories such as the server and apparatus shown in FIG. 2B and FIG. 2C; and

FIG. 7 depicts embodiments of a storage medium such as data storage and memory discussed in conjunction with FIG. 2B, FIG. 2C, and FIG. 6.

FIG. 8 depicts embodiments of a computing platform such as the server(s) discussed in FIG. 2B, the apparatus discussed in FIG. 2C, and the system discussed in FIG. 6.

### DETAILED DESCRIPTION

The following is a detailed description of embodiments depicted in the drawings. The detailed description covers all modifications, equivalents, and alternatives falling within the appended claims.

Simulators significantly reduce the cost and time associated with experimentation. Building prototypes, conducting physical tests, and gathering data in the real world often requires substantial resources and can be time-consuming. Simulators, however, operate in a virtual environment, eliminating the need for physical components and allowing for rapid iteration and testing of countless scenarios within a shorter timeframe. This is particularly valuable for complex processes or those involving expensive equipment, as highlighted in a study by Mckinsey & Company, where simulation reduced development costs for autonomous vehicles by potentially exceeding $1 billion.

Simulators enhance safety and risk mitigation. Testing new processes in the real world can pose risks to personnel, equipment, and even the environment. Simulators provide a safe space to experiment with extreme scenarios, potential failures, and unforeseen circumstances without incurring real-world consequences. This is crucial in fields like biomanufacturing, where experimentation in real-world settings could be disastrous. For instance, simulation-based training in biomanufacturing allows professionals to hone their skills on virtual plants, improving their preparedness for the real-world situations while minimizing risks to actual patients and unit operations.

Simulators facilitate deeper insights and data-driven optimization using machine learning (ML) algorithms. Data that is generated will constantly be fed into the ML algorithm and fine-tune the outputs. The online simulator offers complete control over every variable, allowing researchers to isolate specific factors and analyze their individual and combined effects on the process. This level of granularity and repeatability enables a profound understanding of process dynamics and facilitates data-driven optimization for improved performance and efficiency. A study by AnyLogic emphasizes this capability, showcasing how simulations helped optimize marketing campaigns, leading to significant cost savings and improved effectiveness.

In sum, simulators are not simply substitutes for real-world experimentation, but rather complementary tools offering crucial advantages. An online simulator will further enable access to internal and external customers. By reducing costs, enhancing safety, and providing deeper insights, simulators will empower researchers and developers to optimize processes efficiently and safely, paving the way for innovation and improvement across various domains.

Embodiments discussed herein describe interactive simulators that use historical data and literature to develop a simulated model for continuous any client specified ribonucleic acid (xRNA) production. The simulator facilitates user input from clients and outputs data, graphs, and development plans for continuous xRNA production, giving insight into the xRNA production.

Embodiments may include an online or cloud-based simulator although embodiments are not limited to online or cloud-based instantiations of the simulators. In some embodiments, an online simulator may be web-based, allowing multiple users across one or more client locations and one or more difference areas of technical expertise to collaborate and input their information about the sequence and other client data to generate a development plan. The development plan may include specifications about the design space, achievable yield, purity, critical process parameters, and critical quality attributes.

In embodiments, the simulator is used in a method of simulating an in vitro transcription reaction. In accordance with these embodiments, the method comprises receiving client input, via an intuitive model such as a large language model (LLM). The inputs may include, for example, a concentration value for one or more of a DNA template, a nucleotide triphosphate, a magnesium ion source, such as magnesium chloride and an RNA polymerase. The nucleotide triphosphate may include one or more of guanosine-5′-triphosphate (GTP), adenosine triphosphate (ATP), cytidine triphosphate (CTP), uridine triphosphate (UTP), pseudouridine triphosphate, dihydrouridine triphosphate, 4-thiouridine, inosine triphosphate, 7-methylguanosine triphosphate, 2,7-dimethylguanosine triphosphate, and/or 2,2,7-trimethylguanosine triphosphate. In some aspects, the client input includes a concentration value for each of GTP, ATP, CTP and UTP. The client input is analyzed by the intuitive model. In aspects, the intuitive model may suggest additional parameters and/or alternative values for client-input parameters. Next, a process model simulates the IVT reaction using the input. The process model may include for example a pre-trained partial least squares (PLS) model, where the model is pre-trained on experimental or historical IVT data. In some aspects, a mechanistic model may be incorporated. The model captures relationships between input parameters of an IVT reaction, such as concentrations of DNA template, nucleotide triphosphates, magnesium ion (Mg2+) and an RNA polymerase, and output parameters, such as mRNA yield, reaction efficiency, and byproduct dsRNA formation. In aspects, the model utilizes simulated annealing, genetic algorithms, and/or gradient-based optimization for parameter optimization to achieve user-defined targets. In aspects, a second or further set of client input data is received in an iterative process of optimization to achieve the user-defined targets, such as increased yield with lower consumption of reagents, thereby lowering costs. In an exemplary process, a user designing an IVT process to produce an mRNA vaccine defines a target yield of 1000 μg/mL mRNA in a 2 mL IVT reaction. Using a GUI, the user inputs the following parameters: DNA template: 1 μg/μL, ATP/CTP/GTP/UTP: initially 7.5 mM each, MgCl₂: 10 mM, T7 polymerase dose: 2 KU/mL. The simulator analyzes the input and predicts a yield of only about 680 μg/mL mRNA, with excess NTPs. The user then adjusts the ATP concentration to 8.5 mM, CTP to 6.5 mM, and MgCl₂ to 11.2 mM. The simulator analyzes the input and predicts a yield of 1012 μg/mL mRNA, while reducing NTP consumption by 8%. This provides a cost-optimized formulation for experimental validation by the end-user.

Embodiments may iteratively simulate the continuous RNA production to generate the one or more experimental outcomes and analyze the client input with a pre-trained intuitive model, based on the one or more experimental outcomes, to predict changes to the client input to improve performance of the continuous RNA production with respect to a user-defined target metric. The user-defined target metric may include a target yield, a target purity, a target quality, a target timeline for production, a target budget, or a combination thereof.

In embodiments, a client may iteratively simulate the continuous RNA production to generate the one or more experimental outcomes and utilize one or more intuitive model(s) to analyze the client input with one or more pre-trained intuitive model(s), based on the one or more experimental outcomes, to suggest changes to the client input to improve performance of the continuous RNA production with respect to a user-defined target metric. In accordance with such embodiments, the pre-trained intuitive intuitive model(s) may interact with the client to obtain changes to the client input after provision of the changes suggested by the pre-trained intuitive model(s), wherein the user-defined target metric includes a target yield, a target purity, a target quality, a target timeline for production, a target budget, or a combination thereof.

In embodiments, the development plan may be generated via simulation modes utilizing historical data for whole xRNA production. The client can then execute a design of experiments (DOE) internally or using third-party resources.

After executing the DOE, the client may interact with the simulator to enter the client data from the DOE into a database for the simulator to generate a new iteration of the development plan to improve the modelling of the whole continuous xRNA production. If one or more targets of the whole continuous xRNA production are achieved in the new iteration of the development plan, the client may interact with simulator to enter additional data including parameters to scale-up the whole continuous xRNA production. On the other hand, if one or more of the targets for production are not met and further optimization is required, the client may perform additional DOE and enter the revised DOE data into the database of the simulator to generate another iteration of the development plan.

For convenience, users associated with a client discussed herein may be referred to a client. Client data may refer to communications by one or more users associated with the client's xRNA process such as writings, papers, chats, texts, values entered for simulation purposes, and/or the like. Client data may include a sequence, a capping technology, a type of RNA, an intended use of the xRNA produced, an intended application for the xRNA produced, a target yield, a target purity, a formulation type, a desired storage condition, a desired storage state, an intended delivery device, a target price point, a patient demographic, an administration type, a dose level and concentration, and/or the like.

Some embodiments may generate a graphical user interface including a dashboard interface and a client data input interface. In some embodiments, the dashboard interface may present a status of data entry for the client data, identify gaps in data that still needs to be entered, identify gaps in data that can be entered increase the robustness of the outcomes in a development plan, identify an experimental design or boundaries to explore, identify development plans based on one or more iterations of the simulation, identify determined or expected yields based on one or more iterations of the simulation, identify determined or expected purities based on one or more iterations of the simulation, identify determined or expected purities based on one or more iterations of the simulation, and/or the like.

In some embodiments, the client data input interface may implement natural language processing in models to interact with clients to obtain client data for entry into a simulator database via, e.g., a chatbot interface. Some embodiments may implement natural language processing in models by vectorizing words in client data and analyzing the client data for input values.

Furthermore, some embodiments of the client data input interface may track client data entered by different users associated with a client. For instance, some embodiments may generate subsets of client data or database nodes entered by a particular user associated with the client to allow a particular user to easily review client data entered by that user into the database for the simulator and/or facilitate collaboration between multiple users related to data entered by that user. In some embodiments, the client data input interface may generate one or more draft nodes for client data entry to facilitate collaboration between multiple users prior to committing the client data to the database nodes used by the simulator.

The combination of the database structure and the one or more models to gather client data and perform iterations of a simulation for continuous xRNA production are referred to herein as simulator logic circuitry. Simulator logic circuitry may comprise a combination of hardware and code such as processor circuitry, hard-coded logic, instructions executable in the processor circuitry, memory for short-term and/or long-term storage of instructions and data, and/or the like.

The simulator logic circuitry may implement a relational database in some embodiments and a graph database in other embodiments. The relational database may include a set of users for client, a set of data groups, a client dataset, and a draft client dataset. A model may comprise a mechanistic algorithm, a data-driven model, a hybrid model optionally with artificial intelligence (AI) and a machine learning (ML) model such as a ML statistical model, a neural network, a natural language processing model, a large language model, and/or the like.

A graph database may comprise nodes with edges and each edge of a node may interconnect the node with another node via one or more models. For instance, in some embodiments, a sequence input group of client data may comprise a first node and a dashboard display group of client data may comprise a second node. The first node may interconnect with the second node through a first model that identifies gaps in data that may need to be filled prior to executing a simulation. In some embodiments, the first model may be trained based on historical data and/or literature related to batches for generation of development plans, advantageously providing insight for the client related to a specified sequence. In further embodiments, the first node may interconnect with the second node through two or more models. A first model or set of models may identify gaps in the client data, a second model or set of models may identify gaps in the client data needed for more robust outcomes, and a third model may generate an experimental design or boundaries to explore in relation to the client data.

In embodiments, the client data input interface may employ application program interfaces (APIs), such as APIs for email programs and/or texting programs, to create an instance of the communication for each user group associated with the communication to facilitate or encourage collaboration between different groups of users of the client. For instance, comments related to the client data entered by a user associated with a first group (in a first technical area of expertise) may cause the simulator logic circuitry to access an API for texting and/or emails to generate and distribute instances of the comments to users associated with a second group (in a second area of expertise). Other embodiments may integrate the simulator logic circuitry with a software application such as an email program, a texting program, a text editor, a word processor, a presentation software application, and/or the like.

In embodiments, the simulator logic circuitry may assist by providing more accurate quotations and developing accurate project timelines. In embodiments, the simulator logic circuitry may help clients to: (1) reduce development time from 6-9 months to 1-3 months; (2) reduce manufacturing time from 2-3 months to about 0.5 month; and/or the like.

Various embodiments may address technical problems such as determining more accurate quotations and developing accurate project timelines; reducing development time; reducing manufacturing time; identifying gaps in data that need to be filled (general information); identifying gaps in data needed to perform robust outcomes; generating an experimental design or boundaries to explore; and/or the like.

Different embodiments may advantageously be accessible, scalable available to anyone with an internet connection, enterprise connection, local network, local server, local workstation, and/or the like, fostering collaboration across teams and locations. Different embodiments may advantageously comprise a compilation of data from historical production, literature, and batches. Different embodiments may advantageously generate data during the simulation with models that have as few experimental variables as possible so that the models are reliable and predictable. For example, source of raw materials, enzymes, purity of enzymes, template DNA, temperature, mixing rates etc. Different embodiments may advantageously enable user interaction (via chatbot, dropdown menus, or forms), access, and data upload of client data related to the client's process for xRNA production.

Different embodiments may advantageously be accessible, scalable simulations available to any client with an internet connection, fostering collaboration across teams and locations. This translates to reduced costs and development time compared to real-world testing, while simultaneously enhancing safety and enabling deeper process insights. Moreover, some embodiments with an online platform democratizes access to simulation tools, fostering innovation across sectors as diverse as healthcare, manufacturing, and education. With continuous updates and seamless integration with existing software, some embodiments of such online simulators become powerful allies for optimizing processes, leading to significant industry-wide advancements.

Several embodiments comprise systems with multiple processor cores such as central servers, modems, routers, switches, servers, workstations, netbooks, mobile devices (Laptop, Smart Phone, Tablet, and the like), and the like.

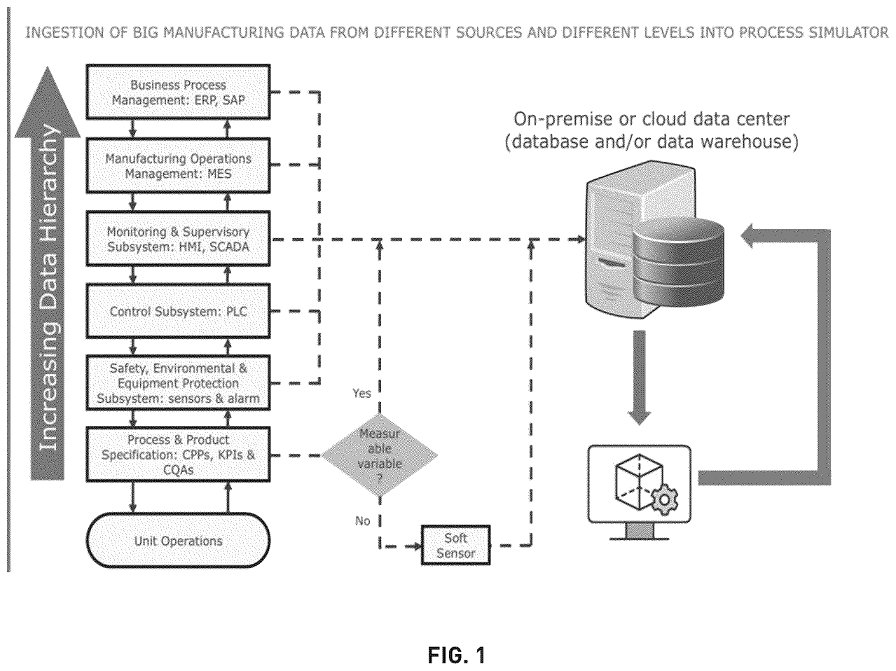

Turning now to the drawings, FIG. 1 to FIG. 4B depict embodiments of systems including servers, networks, data servers, word processing applications, and graphical user interfaces (GUIs) for simulating continuous xRNA production. FIG. 1 depicts a schematic for Hierarchical Ingestion of Manufacturing Data into Process Simulation Platforms. This schematic illustrates how manufacturing data from multiple sources and hierarchical levels—ranging from unit operations and control systems (PLC, sensors, alarms) to enterprise-level systems (SCADA, MES, ERP, SAP)—is ingested into a centralized process simulator via a data center (on-premise or in the cloud). Measurable variables may be directly streamed into the simulator, while non-measurable variables are inferred via soft sensors. The structured data flow enables real-time digital twin implementation, predictive analytics, and data-driven optimization across critical process and product specifications (CPPs, KPIs, CQAs), enhancing decision-making throughout the manufacturing lifecycle.

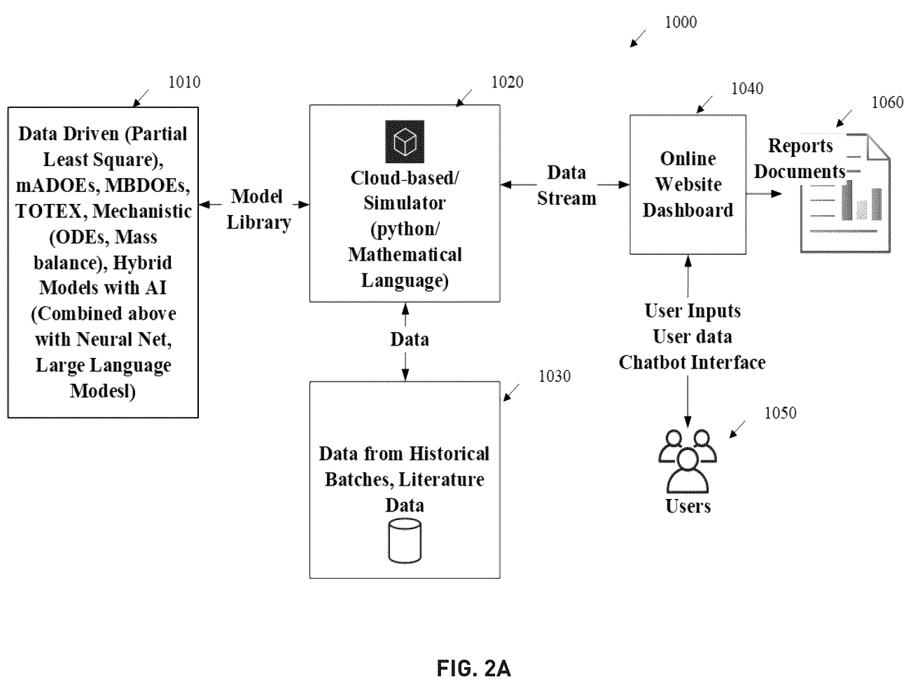

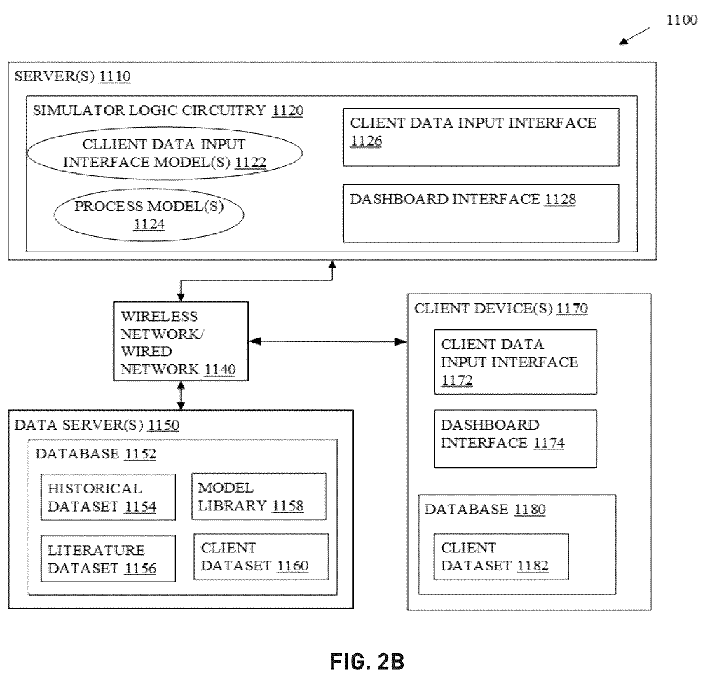

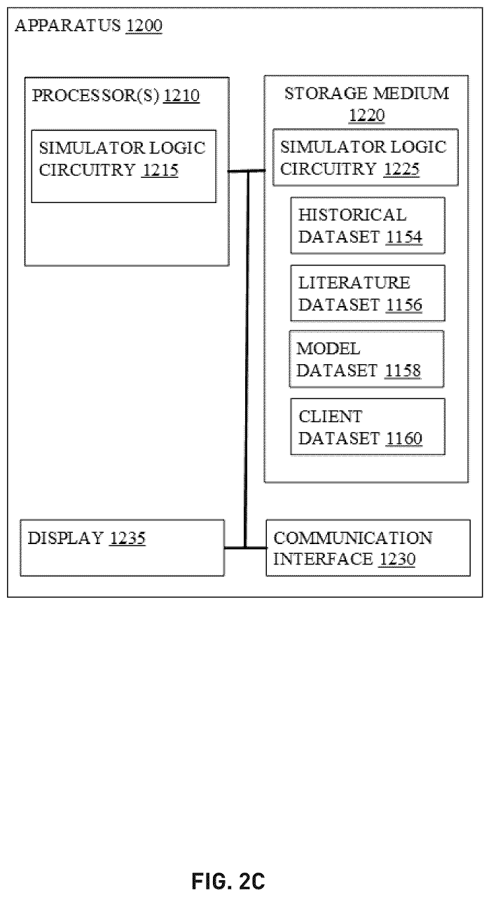

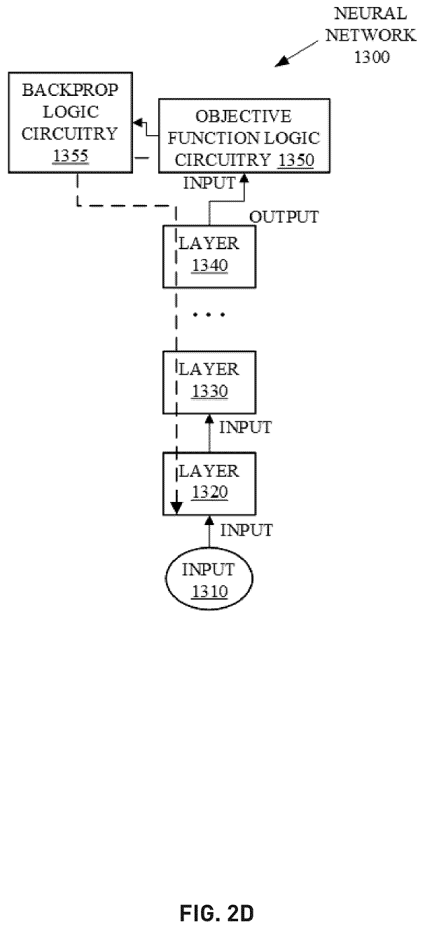

FIG. 2A illustrates a first embodiment of a system 1000 to simulate continuous xRNA production through interaction with users 1050 associated with a client. The system comprises a model library 1010 coupled with a cloud server 1020 comprising simulator logic circuitry. The cloud server 1020 is coupled with a data server 1030 to access datasets including data from historical batches and literature and coupled with a client device 1040 to generate an online website dashboard with a client data input interface to interact with the users 1050 via a conversational model with natural language processing such as a chatbot interface. The client data input interface may also comprise an intuitive interface to answer questions from the users 1050, to direct users 1050 to client data input forms relevant to the client's process, and to direct users to relevant report forms to output various reports related to the client's process.

To illustrate, a client may have a current process for xRNA production or may access the cloud server 1020 via the client device 1040 as part of a process of developing a client's process for xRNA production. In some embodiments, the client may be developing a process and the simulator logic circuitry of the client data input interface on the client device 1040 may interact with a client to determine some basic requirements and definitions of the recipe of the client's process based on data from historical batches and literature relevant to the client's process. For instance, the users 1050 may enter client data to define a sequence for the process and a type of RNA such as mRNA, self-amplifying RNA (saRNA), or circular RNA (cirRNA). Such clients may also enter client data to describe an expected way to administer the xRNA to patients (e.g., as a therapeutic, as a vaccine, or the like), as well as a target of the xRNA such as patients with a rare disease, oncology, or the like. The users 1050 may also enter a target yield and a target purity for the xRNA.

If the client has a current process for xRNA production, the users 1050 may enter further client data to refine the description of the process such as the formulation type and a desired storage condition (such as a temperature for storage). Such clients may also enter client data to describe whether the xRNA produced should be in a powder form or a liquid form as well as the expected delivery device (e.g., vial, microneedle patch, or the like).

With the client data for the process, the simulator logic circuitry of the cloud server 1020 may access the model library to select an intuitive model. The intuitive model may be trained or programed to access data from historical batches and/or literature to gather data about the same or similar production processes and to determine additional client data needed from the users 1050 to improve the likelihood of successfully generating a development plan that will meet the target metrics of the client such as the target yield and the target purity. In some embodiments, the users 1050 may interact with the intuitive model via natural language processing to ask questions about development of the process such as a target price point and/or target timeline needed to meet the target yield or the target purity. The intuitive model may access relevant data from historical batches and/or literature to respond to the users 1050 with a suggested target budget and/or target timeline. In some embodiments, the intuitive model may also suggest changes to client data or additional client data to more likely meet the target budget and/or target timeline such as a target price point, a dose level, and a concentration for the xRNA production. In embodiments, the intuitive models may include large language models (LLMs) such as a OpenAI's chatgpt, Meta's Llama, and/or the like.

With the client data to describe the xRNA production, the simulator logic circuitry of the cloud server 1020 may access the data for historical batches and literature to identify one or more process models to simulate continuous xRNA production. The process models may include one or more models to model each step in the process of continuous production of the xRNA. For instance, the models may include a data-driven model, a mechanistic model, or a hybrid model that combines aspects of both mechanistic and data-driven models with machine learning. Data-driven models are mathematical models based solely on the statistical relationships between data, primarily data obtained or derived from online sensors and offline analysis. Data-driven models are not based on biophysical relationships and may also be referred to as “black-box models.” Data-driven models may include, multiple linear regression (MLR) models, partial least squares regression (PLS) models, partial least squares regression discriminant analysis (PLS-DA) models, structured additive regression (STAR) models, Gaussian process regression (GPR) models, support vector machines regression (SVM) models, model-agnostic DOEs (maDOEs), model-based DOEs (mbDOEs), total expenditure (TOTEX) models, and/or the like.

A mechanistic model refers to a model based on biophysical relationships that have been mathematically elucidated based on a full mathematical understanding of the process and may also be referred to as “white-box models”. Mechanistic models may include mass balance modelling, ordinary differential equations (ODEs) modelling, and/or the like.

Hybrid models with machine learning such as AI may combine mechanistic and data-driven models with neural networks, large language models, and/or the like. In some embodiments, the Hybrid models with AI may include algorithms for carrying out one or more statistical methods selected random forest (RF), neural networks (NNs), deep learning (DL), and/or the like.

The models may utilize the data for historical batches and/or literature that include measurements of the relationship between variables using correlation analysis which relies on establishing correlations between sensor signals, process parameters, and quantity and quality parameters. For example, the extent of the linear relationship may be determined using a Pearson's correlation. Other methods are available to measure nonlinear relationships, for example, Spearman's rank correlation, which is a nonparametric measure of rank correlation reporting the statistical relationship between the rankings of two variables.

After identifying one or more models for simulating the xRNA production, the simulator logic circuitry of the cloud server 1020 may simulate continuous xRNA production based on the client data to model steps including a biological reaction, a filtration operation and/or a chromatography operation. The simulator logic circuitry of the cloud server 1020 may model an interior space of a vessel with inlet and outlet ports and optionally an impeller for fluid recirculation. The simulator logic circuitry of the cloud server 1020 may model a flexible bag or a rigid container for a hold vessel, a release tank, a dilution tank, or a stirred tank reactor. The simulator logic circuitry of the cloud server 1020 may model where the fluid communication between the flow cells, unit operations via a recirculation loop in a vessel external to a process flow path or in-line with the process flow path. The simulator logic circuitry of the cloud server 1020 may model at least one pump. In some embodiments, one or more of the models may perform computational fluid dynamics for each process or sub-process of the steps for the simulation of the continuous xRNA production.

Based on the simulation of the continuous xRNA production, the simulator logic circuitry may generate predicted outcomes for the continuous xRNA production such as expected values for metrics like yield, purity, quality, price points, budgets, process timelines, efficacy, and/or the like. In some embodiments, the simulator logic circuitry of the cloud server 1020 may display expected values, client data, raw material requirements, and other process related information on a dashboard of the display of the client device 1040. In some embodiments, the simulator logic circuitry of the cloud server 1020 may have one or more different report types to report the outcome of the simulation to the users 1050 such as portable document formats (PDFs), text formats, graphs, spreadsheet formats, table formats, and/or the like. In embodiments, one or more conversational models may interact with the users 1050 to design or customize a report format for the users 1050.

In some embodiments, the one or more conversational models may facilitate interaction between various users 1050 or various groups of the users 1050 such as groups of users with different technical backgrounds to support collaboration between the users 1050 of the client. For instance, the one or more conversational models may maintain draft client data entered by users 1050 or groups of users, facilitate commentary by various users 1050, and store commentaries for later review and discussion. The users 1050 may commit or store the draft client data, or portions of the draft client data from one or more of the users into the client data used for a first iteration of the simulation of the continuous xRNA production and/or subsequent iterations of the simulation of the continuous xRNA production. In some embodiments, the simulator logic circuitry of the cloud server 1020 may store amendments or changes to the client data or multiple iterations such that users 1050 may access and compare client data from various iterations of simulations.

Furthermore, one or more intuitive models of the simulation logic circuitry may compare target metrics against expected values generated through one or more iterations of the client data. Based on the comparison, the one or more intuitive models may access data for historical batches and literature and suggest changes to client data or additional client data to fill gaps to adjust the expected values for target metrics in subsequent iterations of the simulations. For instance, in some embodiments, the one or more intuitive models may advantageously suggest client data that can be modified such as the amount of raw materials provided to the process or client data that may be collected by the client through DOEs or through third-party DOEs to improve expected yields, purity, quality, price points, and/or the like.

Such iterations of the simulation may advantageously allow the client to iteratively account for different experiments without having to physically perform the experiments, saving costs and wastes related to materials, time, and money. Such iterations of the simulation may advantageously allow the client to gain insights into the continuous xRNA production via the client's process without having to perform the DOEs. In a race against time in terms of health of patients, such simulations may advantageously be performed in a matter of hours, attaining an ability to begin manufacturing in accordance with the client's process within days or weeks rather than spending months via conventional DOEs.

FIG. 2B illustrates another embodiment of a system 1100. The system 1100 may represent a portion of at least one wireless or wired network 1140 that interconnects server(s) 1110 with data server(s) 1150 and client device(s) 1170. The at least one wireless or wired network 1120 may represent any type of network or communications medium that can interconnect the server(s) 1110, the data server(s) 1150, and the client device(s) 1170, such as a cellular service, a cellular data service, satellite service, other wireless communication networks, fiber optic services, other land-based services, and/or the like, along with supporting equipment such as hubs, routers, switches, amplifiers, and/or the like.

In the present embodiment, the server(s) 1110 and/or the data server(s) 1150 may represent one or more servers owned and/or operated by a company that provides services. In other embodiments, the server(s) 1110 and/or the data server(s) 1150 may represent more than one company that provides services.

The simulator logic circuitry 1120 may comprise code executing on the one or more server(s) 1110. For instance, part of the code, such as the client data input interface model(s) 1122 may execute on a first set of one or more of the server(s) 1110 and another part of the code, such as the process model(s) 1124, may execute on a second set of one or more of the server(s) 1110 to perform iterations of simulations of continuous xRNA production based on the client data in a client dataset 1160.

The client data input interface 1126 may access one or more of client data input interface model(s) 1122 to interact with one or more users of a client via client devices 1170. For instance, the client data input interface 1126 may access a conversational model, an intuitive model, and natural language processing, or a large language model to interact with a user via the client data input interface 1172 (such as a text box on a display) on client device(s) 1170.

The client data input interface model(s) 1122 may comprise algorithms or machine learning models such as natural language models, large language models, neural networks, and/or other machine learning models in the model library 1158 of the database 1152. In embodiments, the client data input interface model(s) 1122 may include conversational models that focus on interaction with users of a client, intuitive models with access to the database 1152, and natural language processing models accessible in the model library 1158. The intuitive models may answer user questions, identify client data gaps, suggest client data values, and suggest additional data gaps based on processing of historical batch data and literature in the historical dataset 1154 and the literature dataset 1156, respectively.

The dashboard interface 1128 may access one or more of process model(s) 1124 to present information about client data, needed client data, suggested client data, and/or expected values generated through one or more iterations of simulations of continuous xRNA production based on client data in the client dataset 1160 via client devices 1170. In some embodiments, the simulator logic circuitry 1120 may interact with a user of a client to customize a display of progress towards entering client data, comparisons of target values with expected values generated through iterations of simulations, lists of expected values resulting from iterations of simulations, and/or the like. In some embodiments, the dashboard interface 1128 may show progress of simulations during execution of process model(s) 1124 for an iteration of a simulation. In embodiments, target values and/or expected values may be presented as numbers, characters, colors, and/or the like, and/or graphs via a dashboard 1174 on a display of the client device(s) 1170.

The process model(s) 1124 may comprise algorithms or machine learning models such as data-driven models, mechanistic models, and hybrid models combined with neural networks or other machine learning models accessible in the model library 1158. In embodiments, the process model(s) 1124 may simulate steps of various aspects of continuous xRNA production defined by client data input in a client dataset 1160 for a client's process. The methodology of the models may depend on the implementation of the client's process. The methodology of the process model(s) 1124 may refer to the model type such as a data-driven models, mechanistic models, and hybrid models combined with AI. As an example, a process model may comprise a linear model such as a logistic regression engine. An example of a non-linear model may be a gradient boosting engine. And an example of a deep learning model may be a deep learning neural network.

In the present embodiment, the data server(s) 1150 may comprise a set of one or more servers dedicated to storage of data for access and use by the one or more server(s) 1110. In some embodiments, the data may be stored and maintained by the one or more data server(s) 1150 in a database 1152 with one or more different types of data structures such as a relational database, a graph database, and/or the like.

In some embodiments, the simulator logic circuitry 1120 may retrieve part of or all the historical dataset 1154, literature dataset 1156, and/or client dataset 1160 to store locally within the server(s) 1110 for use as training and testing datasets and designate portions of the datasets for training data and portions of the datasets for testing data for one or more machine learning models. In some embodiments, the simulator logic circuitry 1115 may access the datasets from the data server(s) 1150 as needed and may cache some of the datasets locally in the server(s) 1110.

In some embodiments, the training dataset and testing dataset may include multiple peer-reviewed historical batches and peer-reviewed literature to train or continue to train machine learning models for client data input model(s) 1122 such as intuitive models and/or process model(s) 1124. For instance, the process model(s) 1124 may train to associate simulation data such as target values with expected values generated via iterations of simulations of the continuous xRNA production. As another example, an intuitive model may train with multiple peer-reviewed historical batches and peer-reviewed literature to determine gaps in client data, suggestions for additional client data, and changes to current client data to meet or exceed target metrics and/or other target values.

The client device(s) 1170 may comprise computers, workstations, laptops, smart phones, and/or the like accessible by users associated with a client. In some embodiments, the client device(s) 1170 may include one or more displays to display the client data input interface 1172 and the dashboard 1174 generated by the simulator logic circuitry 1120 of the one or more server(s) 1120. In some embodiments, the client device(s) 1170 may also store data in, e.g., a database 1180, such as draft client data and/or client data committed as simulation data that is stored in the client dataset 1160.

FIG. 2C depicts an embodiment for an apparatus 1100 such as one of the server(s) 1010 shown in FIG. 2A. The apparatus 1200 may be a computer in the form of a smart phone, a tablet, a notebook, a desktop computer, a workstation, or a server. The apparatus 1200 can combine with any suitable embodiment of the systems, devices, and methods disclosed herein. The apparatus 1200 can include processor(s) 1210, a non-transitory storage medium 1220, communication interface 1230, and a display device 1235. The processor(s) 1210 may comprise one or more processors, such as a programmable processor (e.g., a central processing unit (CPU)). The processor(s) 1210 may comprise processing circuitry to implement simulator logic circuitry 1215 such as the simulator logic circuitry 1120 in FIG. 2B.

The processor(s) 1210 may operatively couple with a non-transitory storage medium 1220. The non-transitory storage medium 1220 may store logic, code, and/or program instructions executable by the processor(s) 1210 for performing one or more instructions including the simulator logic circuitry 1225. The non-transitory storage medium 1220 may comprise one or more memory units (e.g., removable media or external storage such as a secure digital (SD) card, random-access memory (RAM), a flash drive, a hard drive, and/or the like). The memory units of the non-transitory storage medium 1220 can store logic, code and/or program instructions executable by the processor(s) 1210 to perform any suitable embodiment of the methods described herein. For example, the processor(s) 1210 may execute instructions such as instructions of the simulator logic circuitry 1225 causing one or more processors of the processor(s) 1210 represented by the simulator logic circuitry 1215 to interact with users of a client to obtain client data and simulate one or more iterations of continuous xRNA production based on the client data in the client dataset 1160.

The non-transitory storage medium 1220 may store code and data for the simulator logic circuitry 1225, the historical dataset 1154, the literature dataset 1156, the model dataset 1158, and store the client dataset 1160 as shown in FIG. 2B. The simulator logic circuitry 1225 may comprise a complete set of code, whereas the simulator logic circuitry 1215 may comprise portions of the simulator logic circuitry 1225 accessed by the processor(s) 1210 for execution.

The processor(s) 1210 may couple to a communication interface 1230 to transmit and/or receive data from one or more external devices (e.g., a terminal, display device, a smart phone, a tablet, a server, a printer, or other remote device). The communication interface 1230 includes circuitry to transmit and receive communications through a wired and/or wireless media such as an Ethernet interface, a wireless fidelity (Wi-Fi) interface, a cellular data interface, and/or the like. In some embodiments, the communication interface 1230 may implement logic such as code in a baseband processor to interact with a physical layer device to transmit and receive wireless communications such as client data from a server. For example, the communication interface 1230 may implement one or more of local area networks (LAN), wide area networks (WAN), infrared, radio, Wi-Fi, point-to-point (P2P) networks, telecommunication networks, cloud communication, and the like.

FIG. 2D depicts an embodiment of a neural network (NN) 1300 of a simulator logic circuitry, such as one or more of the hybrid models, intuitive models, conversational models, and/or natural language processing models of the model dataset 1160 shown in FIG. 2B to FIG. 2C. The NN 1300 may comprise as a deep neural network (DNN).

A DNN is a class of artificial neural network with a cascade of multiple layers that use the output from the previous layer as input. An example of a DNN is a recurrent neural network (RNN) where connections between nodes form a directed graph along a sequence. A feedforward neural network is a neural network in which the output of each layer is the input of a subsequent layer in the neural network rather than having a recursive loop at each layer.

Another example of a DNN is a convolutional neural network (CNN). A CNN is a class of deep, feed-forward artificial neural networks. A CNN may comprise of an input layer and an output layer, as well as multiple hidden layers. The hidden layers of a CNN typically consist of convolutional layers, pooling layers, fully connected layers, and normalization layers.

The NN 1300 comprises an input layer 1310, and three or more layers 1320 and 1330 through 1340. The input layer 1310 may comprise input data that is training data for the NN 1300. The input layer 1310 may provide data in the form of tensor data to the layer 1320. The tensor data may include a vector, matrix, or the like with values associated with each input feature of the NN 1300.

In embodiments, the input layer 1310 is not modified by backpropagation. The layer 1320 may compute an output and pass the output to the layer 1330. Layer 1330 may determine an output based on the input from layer 1320 and pass the output to the next layer and so on until the layer 1340 receives the output of the second to last layer in the NN 1300. Depending on the methodology of the NN 1300, each layer may include input functions, activation functions, and/or other functions as well as weights and biases assigned to each of the input features. The weights and biases may be randomly selected or defined for the initial state of a new model and may be adjusted through training via backwards propagation (also referred to as backpropagation or backprop). When retraining a model with data obtained after an initial training of the model, the weights and biases may have values related to the previous training and may be adjusted through retraining via backwards propagation.

The layer 1340 may generate an output and pass the output to an objective function logic circuitry 1350. The objective function logic circuitry 1350 may determine errors in the output from the layer 1340 based on an objective function such as a comparison of the predicted results against the expected results. For instance, the expected results may be paired with the input in the training data supplied for the NN 1300 for supervised training.

During the training mode, the objective function logic circuitry 1350 may output errors to backpropagation logic circuitry 1355 to backpropagate the errors through the NN 1300. For instance, the objective function logic circuitry 1350 may output the errors in the form of a gradient of the objective function with respect to the input features of the NN 1300.

The backpropagation logic circuitry 1355 may propagate the gradient of the objective function from the top-most layer, layer 1340, to the bottom-most layer, layer 1320 using the chain rule. The chain rule is a formula for computing the derivative of the composition of two or more functions. That is, if f and g are functions, then the chain rule expresses the derivative of their composition f. g (the function which maps x to f(g(x))) in terms of the derivatives of f and g. After the objective function logic circuitry 1350 computes the errors, backpropagation logic circuitry 1355 backpropagates the errors. The backpropagation is illustrated with the dashed arrows.

When operating in inference mode, the simulator logic circuitry, such as the simulator logic circuitry 1215 shown in FIG. 2C, may receive feedback and, in some embodiments, the feedback may be in the form of word vectors or a sentence analysis of the word vectors for each of one or more sentences. If the feedback is negative, the backpropagation may attribute an error to the replacement. If the feedback is positive, the backpropagation may reinforce or bias selection of the replacement within the layers of the NN 1300.

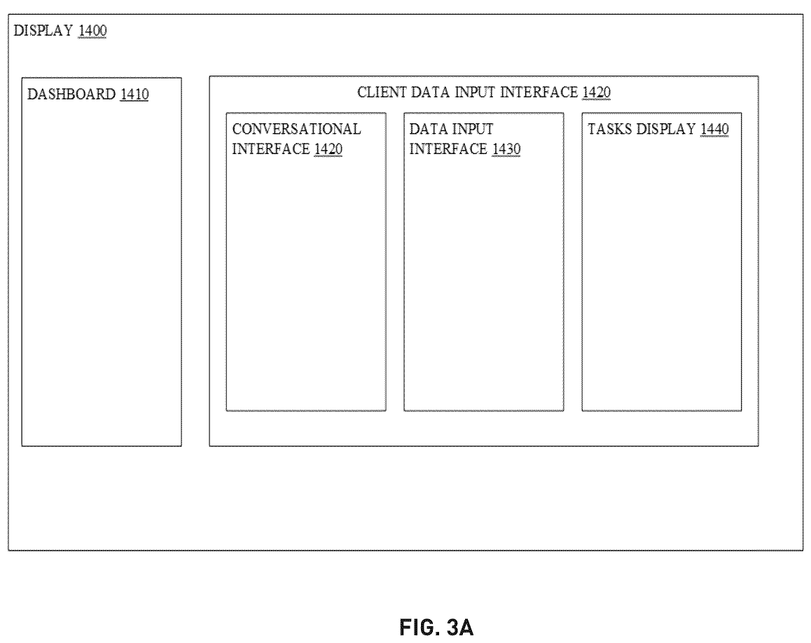

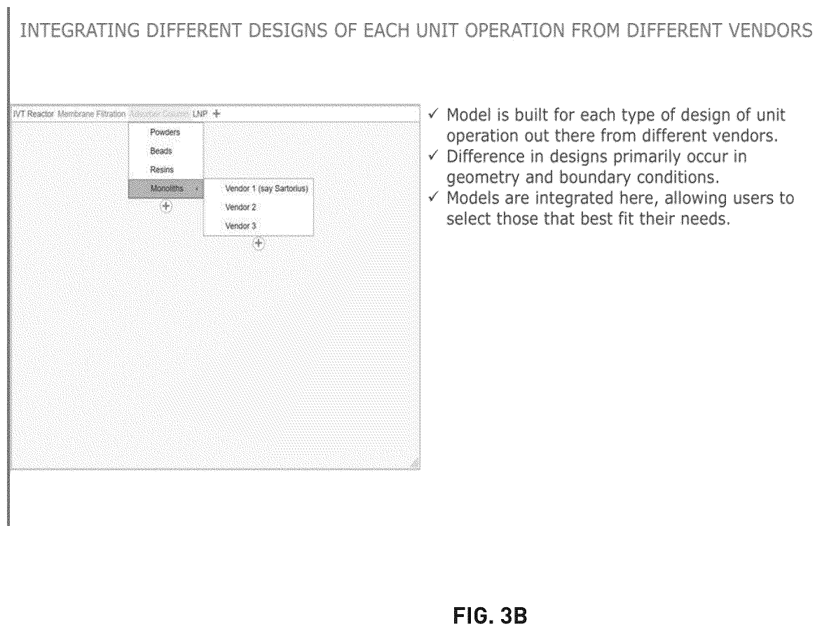

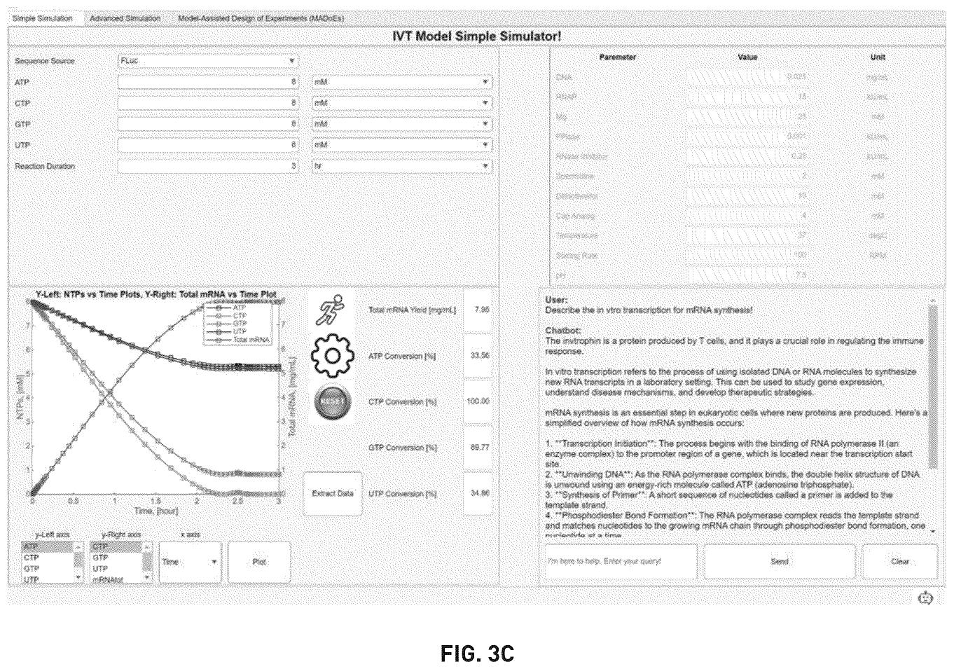

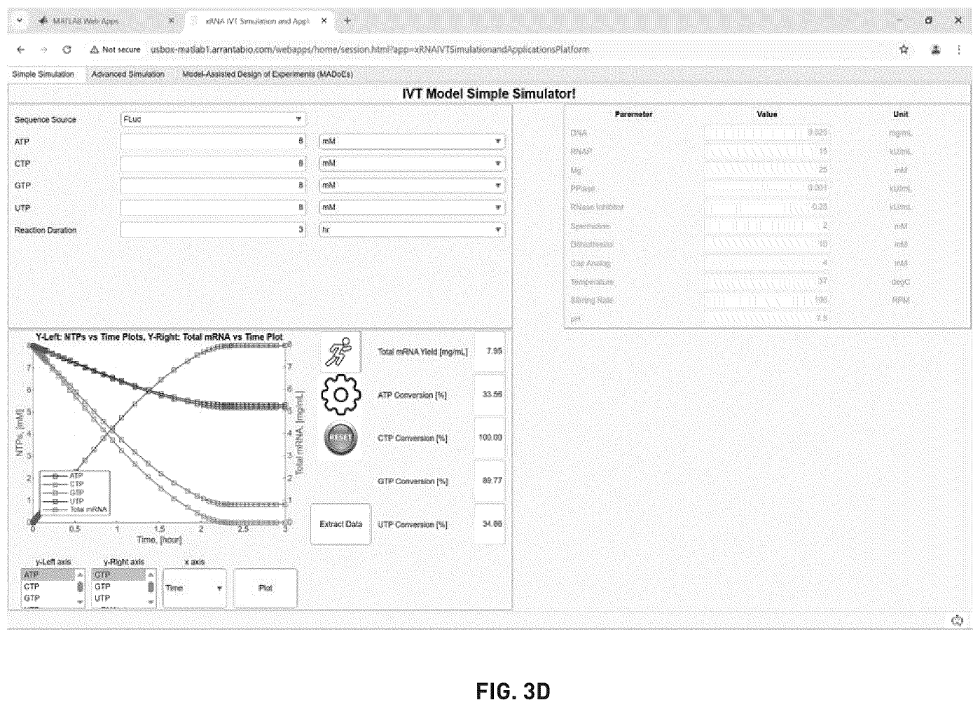

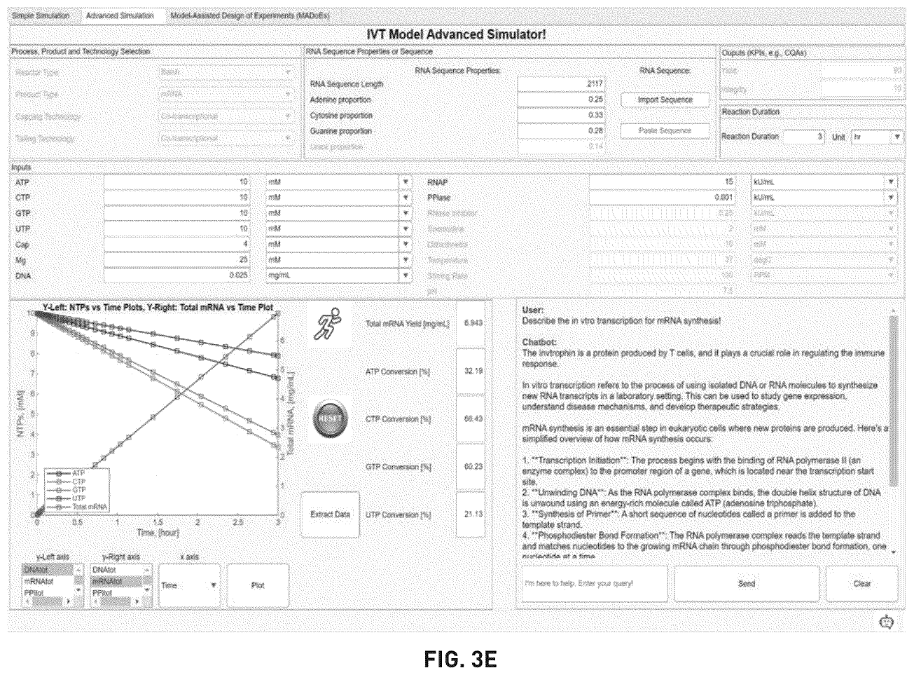

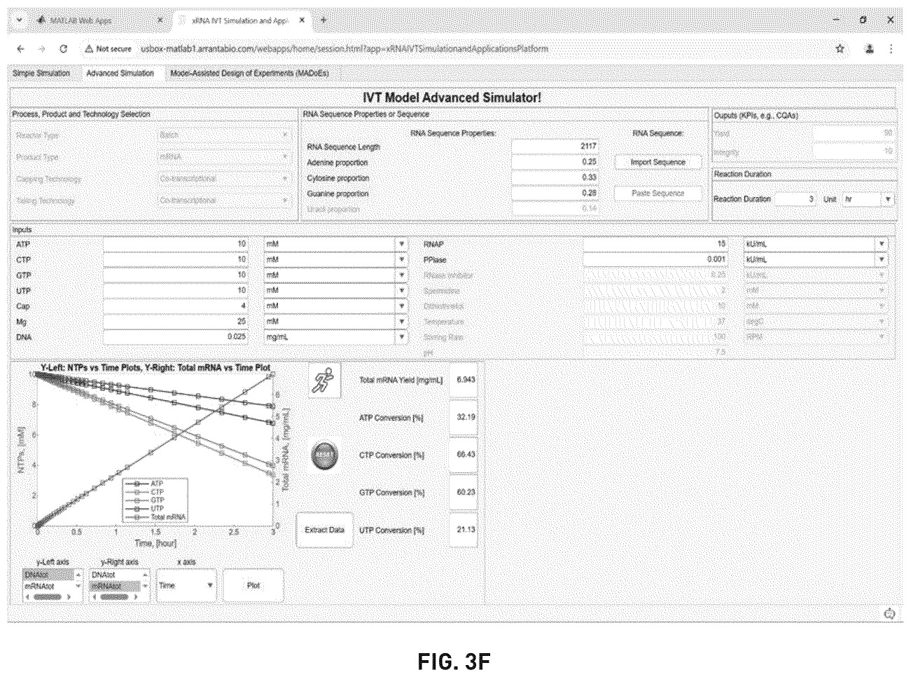

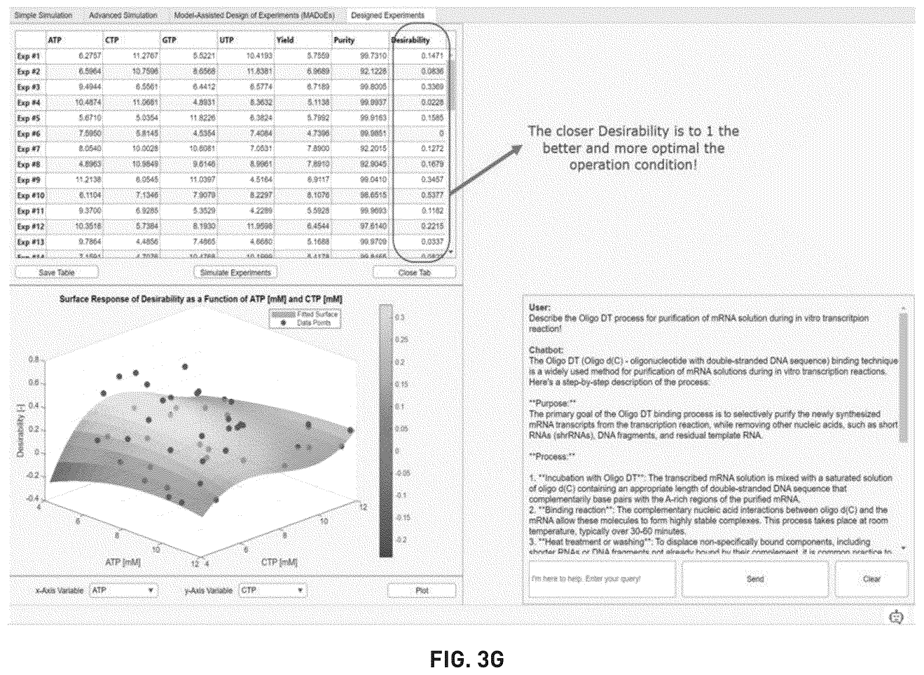

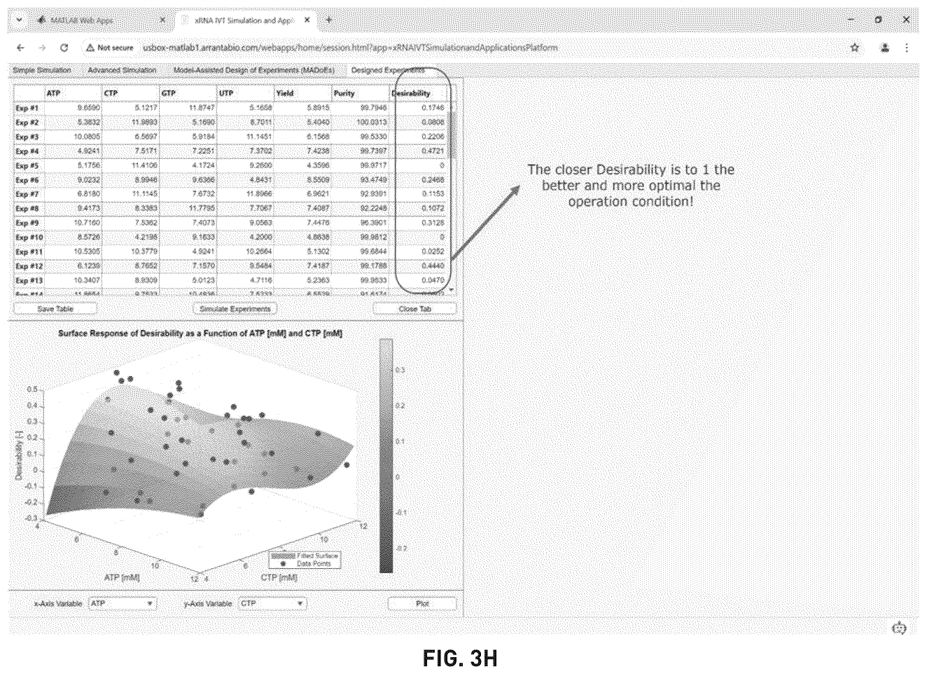

FIG. 3A depicts an embodiment of a graphical user interface (GUI) on a display 1400 for client interaction with simulator logic circuitry, such as the simulator logic circuitry 1120 and 1225 illustrated in FIG. 2B to FIG. 2C and the display 1235 shown in FIG. 2C. The graphical user interface of display 1400 illustrates a dashboard 1410 and a client data input interface 1420, which may be a display of a client device that is generated by simulator logic circuitry on a remote server. The dashboard 1410 may display client data entered, client data to be entered, client data suggested by an intuitive model, target values including metrics for a simulation, and expected values generated by an iteration of a simulation.

The client data input interface 1420 may comprise a conversational interface 1420, a data input interface 1430, and a task display 1440. The conversational interface 1420 may comprise a display of a text of a conversation with a conversational model of the simulator logic circuitry. The data input interface 1430 may include a form for client data input. In some embodiments, the data input interface 1430 may include client data entered. In some embodiments, the data input interface 1430 may include client data to be entered and or data suggested to be entered.

The task display 1440 may identify a set of tasks to perform or suggested to be performed by the intuitive model of the simulator logic circuitry prior to performance of a subsequent iteration of the simulation for xRNA production.

FIG. 3B illustrates a GUI of a simulation platform that enables users to select and simulate different unit operations—such as IVT reactors, membrane filtration, adsorber columns, and LNP formulation—each with multiple design options based on media type (e.g., powders, beads, resins, monoliths) and vendor-specific configurations. These models differ in geometry and boundary conditions and are already embedded within the simulation backend, in some embodiments. By providing a unified interface for selecting and comparing vendor-specific unit designs, the simulation platform supports flexible, accurate, and vendor-aware process modeling for end-to-end biomanufacturing workflows. From a pull-down menu, customers maybe able to:

Select the unit operations they are interested in simulating (e.g., IVT Reactor, Membrane Filtration, Adsorber Column, LNP).

Within each unit operation, choose from a range of material types or media—for example:
For adsorber columns: Powders, Beads, Resins, and Monoliths.

Upon selecting a material (e.g., Monoliths), a vendor-specific sub-menu will appear, allowing the customer to:
Choose a specific design configuration from different vendors.
Each design differs in geometry, wall structure, boundary conditions, which may already be encoded into the backend model in some embodiments.

Once a vendor design is selected, the simulator may:
Automatically load the appropriate geometry and physical parameters
Simulate the unit operation behaviors under selected process conditions.
Provide results such as flow distribution, process key performance indicators (KPIs), and critical quality attributes (CQAs).

FIG. 3C illustrates a GUI of a simplified simulator interface that allows users to explore the dynamics of mRNA synthesis via in vitro transcription (IVT) using minimal input parameters such as initial nucleotide concentrations (ATP, CTP, GTP, UTP), sequence source, and reaction duration. The interface computes and visualizes real-time nucleotide consumption and mRNA accumulation over the course of the reaction. Key outputs include total mRNA yield and nucleotide conversion efficiencies, enabling rapid prototyping and preliminary optimization of IVT conditions with minimal user input. In this embodiment, the chatbot is toggled on and used. In further embodiments, the chatbot may be toggled off.

FIG. 3D illustrates a web version of FIG. 3C with chatbot toggled off and unused to demonstrate control over chatbot visibility and continuously obstructing background UI components. In further embodiments, the chatbot may be toggled on. The simulation logic circuitry includes one or more intuitive model(s) and one or more process model(s) to help users optimize a process. In embodiments, the optimization process may include iterations of analysis and simulation. Prior to or during an initial simulation, the one or more intuitive model(s) may analyze the client input information for a process based on training and/or access of historical batches and literature. The intuitive model(s) may offer suggest changes to client data including parameter values, data values, and the like, suggest additional parameter values, data values, and/or the like, and suggest additional client data that the user should enter or capture during the process to facilitate optimization.

After the initial simulation, the one or more intuitive model(s) may analyze the information generated during the process simulation and the results of the simulation in addition to historical batches and literature to determine one or more predictions about the process simulation. For instance, the intuitive model(s) may suggest supplying additional raw materials to increase the speed of a process or to increase a concentration level resulting from the process.

The users may adjust the inputs for the process simulation based on one or more of the suggestions to optimize the process. After adjusting the inputs, the process model(s) may simulate the process based on the adjusted inputs to determine the results of the process.

In embodiments, the process model(s) may simulate an IVT process with PLS models or mechanistic models. For instance, a MATLAB GUI simulator may integrate Partial Least Squares (PLS) models trained on experimental or historical IVT data. These process models capture relationships between the inputs: concentrations of DNA template, NTPs, Mg²⁺, etc and outputs: mRNA yield, reaction efficiency, byproduct dsRNA formation, etc. An optimization workflow may iterate a process of receiving user inputs for experimental parameters (e.g., initial NTPs, pH, temperature, DNA dose), which may include suggestions based on analyses by the intuitive model(s) and the process model(s) may output predicted RNA yield and resource consumption. The end user can iterate by either adjusting inputs (e.g., ATP: GTP ratio, Mg²⁺ level) or entering or accepting suggestions from the intuitive models prior to each iteration of the process simulation to maximize predicted yield or reduce reagent cost. In embodiments, an embedded optimizer (intuitive model(s)) uses simulated annealing, genetic algorithms, or gradient-based optimization to automate the parameter tuning to achieve targets (e.g., maximum yield at lowest cost). Consider a case study where an end-user designing a mRNA process for a new vaccine targets 1000 μg/mL mRNA yield in a 2 mL IVT reaction. Using a GUI for one or more interface model(s), the end user may input the following parameters: DNA template: 1 μg/μL, ATP/CTP/GTP/UTP: initially 7.5 mM each, MgCl₂: 10 mM, T7 polymerase dose: 2 KU/mL. The process model(s) may predict only ˜680 μg/mL mRNA, with excess NTPs. The end user can adjust ATP concentration to 8.5 mM, CTP to 6.5 mM, and MgCl₂ to 11.2 mM, optionally based on suggestions from the intuitive model(s) and perform an additional iteration of process simulation. After the additional iteration of the process simulation, the process model(s) may predict: 1012 μg/mL mRNA, while reducing NTP consumption by 8%. Based on these results from the process simulation, the end user has a cost-optimized formulation for experimental validation.

FIG. 3E illustrates a GUI of an interactive simulation interface that enables users to model and analyze the in vitro transcription (IVT) process for mRNA production. Users may input key parameters such as nucleotide concentrations (ATP, CTP, GTP, UTP), enzyme levels, DNA template concentration, and RNA sequence properties. The simulator may output key performance indicators (KPIs) including total mRNA yield and nucleotide conversion percentages. Furthermore, the plot on a GUI display may visualize real-time consumption of nucleotides and accumulation of mRNA over time, helping users optimize conditions for maximum transcription efficiency.

FIG. 3F illustrates a web version of FIG. 3E with chatbot toggled off and unused to demonstrate control over chatbot visibility and continuously obstructing background/future UI components. In further embodiments, the chatbot may be toggled on.

FIG. 3G illustrates a GUI of a simulator (desktop/installable version) displays a matrix of experimental conditions varying nucleotide concentrations (ATP, CTP, GTP, UTP) and the corresponding mRNA yield, purity, and computed desirability score. Desirability values range from 0 to 1, with values closer to 1 indicating more optimal operational conditions. The 3D surface plot visualizes the response surface of desirability as a function of ATP and CTP concentrations, aiding users in identifying optimal regions in the design space, which can inform further experimental investigation or validation, ultimately leading to enhanced process performance.

In embodiments, the simulator includes a Design of Experiments (DoE) process model for each unit operation in the RNA manufacturing process, including for example IVT, filtration, and chromatography unit operations. Aspects of the DoE process model are informed by mechanistic, data-driven or hybrid process models pre-trained on specific unit operations, for example a diafiltration process. In an exemplary implementation, a user seeks to optimize transmembrane pressure (TMP) and investigate fouling of a tangential flow filtration (TFF) membrane in a filtration unit operation of the manufacturing process. Using a GUI for interface model(s), the user inputs one or more parameters selected from number of diavolumes, membrane molecular weight cut off (MWCO), and retentate flow rate. The process model(s) of the simulator analyzes the user input and returns a set of predicted outputs such as final RNA concentration, residual NTPs and impurity levels, and a shear stress profile. Based on these results, the intuitive model(s) may suggest changes to the client data, additional data, suggested values for additional data, and/or the like. The user adjusts aspects of the input, such as membrane pore size number of diavolumes, as needed, in order to reach a target parameter, such as impurity clearance, for example an impurity clearance of greater than 98%. In further iterations or the process simulation, the user may continue to adjust inputs (optionally based on suggestions from the intuitive model(s)) to maintain the target parameter(s) while improving process efficiency, for example by reducing processing time. The results inform scale-down experiments to verify performance.

In the present embodiment, the chatbot is toggled on. In further embodiments, the chatbot may be toggled off.

FIG. 3H illustrates a web version of FIG. 3G with chatbot toggled off and unused to demonstrate control over chatbot visibility and continuously obstructing background/future UI components. In further embodiments, the chatbot may be toggled on.

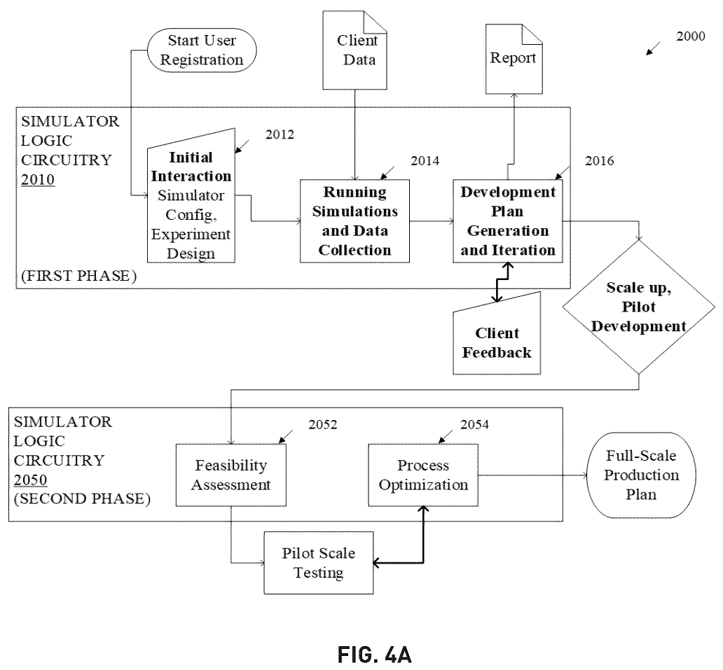

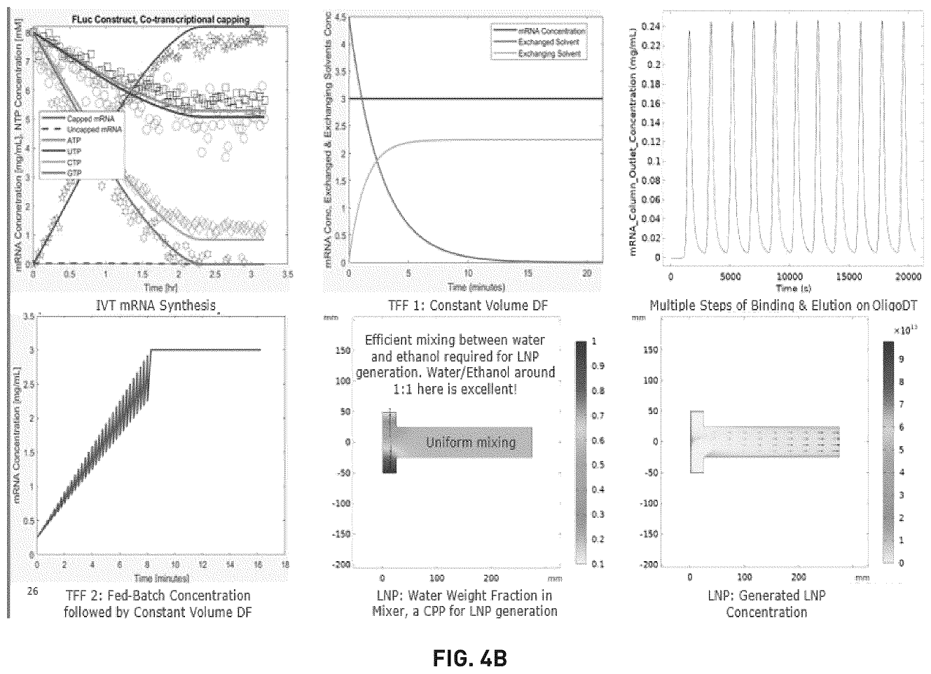

FIG. 4A depicts an embodiment of simulator logic circuitry 2000, such as the simulator logic circuitry shown in FIG. 2B and FIG. 2C. In the present embodiment, the simulator logic circuitry 2000 comprises a first phase simulator logic circuitry 2010 to begin from pre-clinical xRNA process to a development plan for pilot production and a second phase simulator logic circuitry 2050 to develop the pilot production into a full-scale, continuous xRNA production plan. A client may begin by registering one or more users associated with the client with the first phase simulator logic circuitry 2010 to give access to the users for entry of client data and configure parameters for the experimental design at an initial interaction 2012 step.

After the registration process, the users may begin a running simulation and data collection 2014 step through access to data entry forms via a GUI such as the GUIs shown in FIG. 3D through FIG. 3F. FIG. 3D through FIG. 3F illustrate menus for an IVT process.

The data entry forms may be accessible in the GUI through pull down menus, directories, input form listings, and/or the like, or through access to the text box with a chatbot type of interface for a conversational model, an intuitive model, and a natural language processing model. If the user chooses, the user may ask questions in the text box or the conversation model may ask the user questions about the client's process to obtain client information that the intuitive model may use to determine which client data input form or forms that may be most appropriate for the user. For instance, the intuitive model may direct the user to a basic client process form that requests client data about design space, target yield, target purity, other critical process parameters (e.g., a sequence, a type of xRNA, etc.) and critical quality attributes (dose concentrations, delivery device, and/or the like), for the client process.

In some embodiments, if the client is in the development stage of a process, the intuitive model may access data for historical batches and literature to ask the client questions about the client process and suggest some of the client data values for the process. In some embodiments, the intuitive model may access data for historical batches and literature to request that the users obtain other client data needed and/or suggested for a first iteration of a simulation of the client process for continuous xRNA production. In embodiments, the multiple users of the client may have a display with a dashboard on client devices such as workstations, laptops, smart phones, etcetera, owned by the client and connected to the first phase simulator logic circuitry 2010 via a web browser interface or a local client application for the first phase simulator logic circuitry 2010.

Once the client data needed for a simulation is entered, the first phase simulator logic circuitry 2010 may execute a first iteration of the simulation on process models selected by the intuitive model based on data for historical batches and literature such as the models discussed in conjunction with FIG. 4B to generate the plots discussed in conjunction with FIG. 4B. The first iteration of the simulation may produce expected outcomes of a pilot production in the form of a development plan. The development plan may include protocols for a continuous, xRNA production based on the client data. The expected outcomes may include expected yields, purity, process time, and/or the like that may meet or exceed the target values for the yields, purity, process time, and/or the like or that may fall short of the target values. If the expected users may proceed to scale-up with pilot development and access the second phase simulator logic circuitry 2050.

FIG. 4B shows examples of unit operation process parameters simulated; these include IVT mRNA synthesis, tangential flow filtration (TFF 1) solvent exchange, Oligo dT purification, TFF fed-batch concentration followed by constant volume DF; lipid nanoparticle formulation.

On the other hand, if one or more of the expected values do not meet or exceed the target values, the users for the client may interact with intuitive model and provide client feedback to obtain suggestions from the intuitive model as to how the improve the expected values during a subsequent iteration of the simulation. The intuitive model may access and analyze the historical data and literature to suggest one or more changes to the client data or to suggest additional client data to improve the expected values. Thereafter, a subsequent iteration may be executed based on the client feedback. FIG. 3G and FIG. 3H illustrate this for an IVT process (Multi-Objective Optimization Interface for In Vitro Transcription (IVT) Process).

In some embodiments, once an iteration of the simulation is completed, the first phase simulator logic circuitry 2010 may generate one or more reports and a development plan based on the latest iteration of the simulation.

After one or multiple iterations of the simulation, the users may interact with models of the second phase simulator logic circuitry 2052 to perform a feasibility assessment 2052. The feasibility assessment 2052 may involve interaction with the users of the client assess the feasibility for full-scale production as defined by the users. During the feasibility assessment, the same or a different intuitive model may interact with the users of the client via the conversational model and a natural language processing model to determine client data for the full-scale production. Such a scale-up may involve performance of pilot scale testing performed by the client or a third party based on the development plan generated by the first phase simulation logic circuitry 2010.

After the pilot scale testing, the users may enter client data or modify the client data used for the latest iteration of the simulation for process optimization 2054 to optimize the client data for the full-scale production. The second phase simulator logic circuitry 2050 may then generate a full-scale production plan for continuous xRNA production.

FIG. 4B illustrates a GUI of plots with different unit operation process parameters simulated with the simulator logic circuitry discussed herein. The GUI may allow the models and corresponding plots to be integrated into one or more displays for the GUI. Once customized, the customer may provide different inputs, the simulator logic circuitry may generate new plots, and the GUI may present the new plots on the one or more displays.

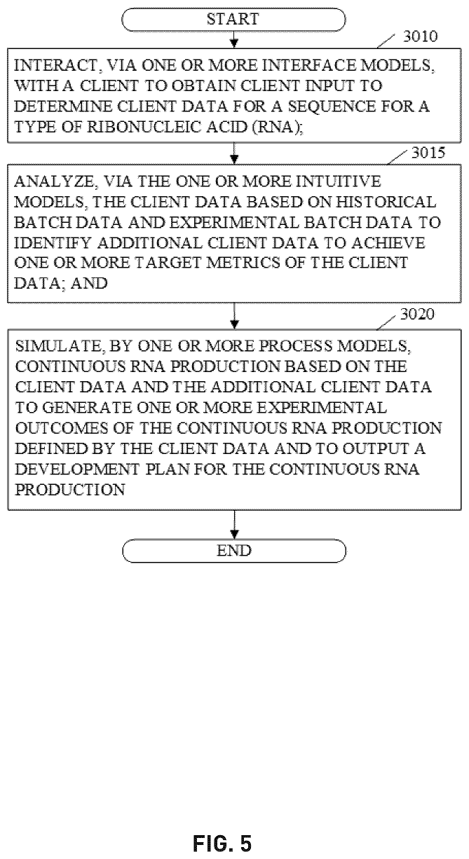

FIG. 5 depicts a flowchart of embodiments of process to simulate continuous xRNA production based on a client process to generate a development plan and scale-up the development plan from pilot development to a full-scale production plan, by simulator logic circuitry, such as the simulator logic circuitry shown in FIG. 2B to FIG. 2C and FIG. 4A. The process starts with interacting, via one or more interface models, with a client to obtain client input to determine client data for a sequence for a type of ribonucleic acid (RNA) (element 3010). In some embodiments, the one or more process models may comprise one or more machine learning models, one or more data-driven models, one or more mechanistic models, one or more hybrid models, and/or a combination thereof to simulate steps of a continuous RNA production process. The one or more interface models may comprise a natural language processing model, a conversational language model, a large language model, or a combination thereof. In some embodiments, the one or more interface models comprises a large language model to interact with the client.

After interacting with the users, the process may proceed to analyze, via the one or more intuitive models, the client data based on historical batch data and experimental batch data to identify additional client data to achieve one or more target metrics of the client data (element 3015). In some embodiments, the experimental batch data may reside in peer-reviewed literature.

Based on the analysis, the process may simulate, by one or more process models, continuous RNA production based on the client data and the additional client data to generate one or more experimental outcomes of the continuous RNA production defined by the client data, and to output a development plan for the continuous RNA production (element 3020). In some embodiments, the one or more process models are based on historical batch data, peer-reviewed experimental batch data, peer-reviewed literature, or a combination thereof to simulate steps of the continuous RNA process to predict the one or more experimental outcomes. In some embodiments, the one or more intuitive models may suggest that the amendments of the client data and the additional client data to add more data or revise current data after each iteration of simulation. Based on client feedback, the simulator logic circuitry may perform additional iterations of the simulation and revisions of client data according to client feedback until one or more target metrics are met. In some embodiments, the target metric may comprise a target yield, a target purity, a target quality, a target timeline for production, a target budget, or a combination thereof.

After attaining expected values that meet or exceed the target metrics, the simulator logic circuitry may, via one or more scale-up models, scale-up the development plan to full commercial production plan.

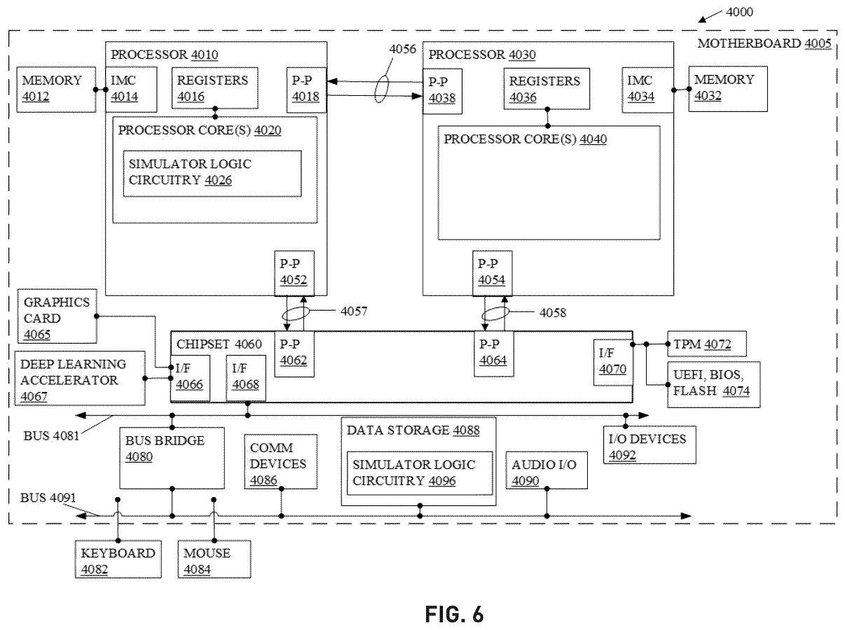

FIG. 6 illustrates an embodiment of a system 4000 such as a server of the server(s) 1110 shown in FIG. 2B or the apparatus 1200 shown in FIG. 2C. The system 4000 is a computer system with multiple processor cores such as a distributed computing system, supercomputer, high-performance computing system, computing cluster, mainframe computer, mini-computer, client-server system, personal computer (PC), workstation, server, portable computer, laptop computer, tablet computer, handheld device such as a personal digital assistant (PDA), or other device for processing, displaying, or transmitting information. Similar embodiments may comprise, e.g., a smart phone or other cellular phone, an external storage device, or the like. Further embodiments implement larger scale server configurations. In other embodiments, the system 4000 may have a single processor with one core or more than one processor. Note that the term “processor” refers to a processor with a single core or a processor package with multiple processor cores.

As shown in FIG. 6, system 4000 comprises a motherboard 4005 for mounting platform components. The motherboard 4005 is a point-to-point interconnect platform that includes a first processor 4010 and a second processor 4030 coupled via a point-to-point interconnect 4056 such as an Ultra Path Interconnect (UPI). In other embodiments, the system 4000 may be of another bus architecture, such as a multi-drop bus. Furthermore, each of processors 4010 and 4030 may be processor packages with multiple processor cores including processor core(s) 4020 and 4040, respectively. While the system 4000 is an example of a two-socket (2S) platform, other embodiments may include more than two sockets or one socket. For example, some embodiments may include a four-socket (4S) platform or an eight-socket (8S) platform. Each socket is a mount for a processor and may have a socket identifier. Note that the term platform refers to the motherboard with certain components mounted such as the processors 4010 and the chipset 4060. Some platforms may include additional components and some platforms may only include sockets to mount the processors and/or the chipset.

The first processor 4010 includes an integrated memory controller (IMC) 4014 and point-to-point (P-P) interconnects 4018 and 4052. Similarly, the second processor 4030 includes an IMC 4034 and P-P interconnects 4038 and 4054. The IMC's 4014 and 4034 couple the processors 4010 and 4030, respectively, to respective memories, a memory 4012 and a memory 4032. The memories 4012 and 4032 may be portions of the main memory (e.g., a dynamic random-access memory (DRAM)) for the platform such as double data rate type 3 (DDR3) or type 4 (DDR4) synchronous DRAM (SDRAM). In the present embodiment, the memories 4012 and 4032 locally attach to the respective processors 4010 and 4030. In other embodiments, the main memory may couple with the processors via a bus and shared memory hub.

The processors 4010 and 4030 comprise caches coupled with each of the processor core(s) 4020 and 4040, respectively. In the present embodiment, the processor core(s) 4020 of the processor 4010 include a simulator logic circuitry 4026 such as the simulator logic circuitry 1120 shown in FIG. 2B. The simulator logic circuitry 4026 may represent circuitry configured to perform an interactive simulator for continuous xRNA production within the processor core(s) 4020 or may represent a combination of the circuitry within a processor and a medium to store all or part of the functionality of the simulator logic circuitry 4026 in memory such as cache, the memory 4012, buffers, registers, and/or the like. In several embodiments, the functionality of the simulator logic circuitry 4026 resides in whole or in part as code in a memory such as the simulator logic circuitry 4096 in the data storage unit 4088 attached to the processor 4010 via a chipset 4060 such as the simulator logic circuitry 1110 shown in FIG. 2B. The functionality of the simulator logic circuitry 4026 may also reside in whole or in part in memory such as the memory 4012 and/or a cache of the processor. Furthermore, the functionality of the simulator logic circuitry 4026 may also reside in whole or in part as circuitry within the processor 4010 and may perform operations, e.g., within registers or buffers such as the registers 4016 within the processor 4010, registers 4036 within the processor 4030, or within an instruction pipeline of the processor 4010 or the processor 4030.

In other embodiments, more than one of the processor 4010 and 4030 may comprise functionality of the simulator logic circuitry 4026 such as the processor 4030 and/or the processor within the deep learning accelerator 4067 coupled with the chipset 4060 via an interface (I/F) 4066. The I/F 4066 may be, for example, a Peripheral Component Interconnect-enhanced (PCI-e).

The first processor 4010 couples to a chipset 4060 via P-P interconnects 4052 and 4062 and the second processor 4030 couples to a chipset 4060 via P-P interconnects 4054 and 4064. Direct Media Interfaces (DMIs) 4057 and 4058 may couple the P-P interconnects 4052 and 4062 and the P-P interconnects 4054 and 4064, respectively. The DMI may be a high-speed interconnect that facilitates, e.g., eight Giga Transfers per second (GT/s) such as DMI 3.0. In other embodiments, the processors 4010 and 4030 may interconnect via a bus.

The chipset 4060 may comprise a controller hub such as a platform controller hub (PCH). The chipset 4060 may include a system clock to perform clocking functions and include interfaces for an I/O bus such as a universal serial bus (USB), peripheral component interconnects (PCIs), serial peripheral interconnects (SPIs), integrated interconnects (I2Cs), and the like, to facilitate connection of peripheral devices on the platform. In other embodiments, the chipset 4060 may comprise more than one controller hub such as a chipset with a memory controller hub, a graphics controller hub, and an input/output (I/O) controller hub.

In the present embodiment, the chipset 4060 couples with a trusted platform module (TPM) 4072 and the unified extensible firmware interface (UEFI), BIOS, Flash component 4074 via an interface (I/F) 4070. The TPM 4072 is a dedicated microcontroller designed to secure hardware by integrating cryptographic keys into devices. The UEFI, BIOS, Flash component 4074 may provide pre-boot code.

Furthermore, chipset 4060 includes an I/F 4066 to couple chipset 4060 with a high-performance graphics engine, graphics card 4065. In other embodiments, the system 4000 may include a flexible display interface (FDI) between the processors 4010 and 4030 and the chipset 4060. The FDI interconnects a graphics processor core in a processor with the chipset 4060.

Various I/O devices 4092 couple to the bus 4081, along with a bus bridge 4080 which couples the bus 4081 to a second bus 4091 and an I/F 4068 that connects the bus 4081 with the chipset 4060. In one embodiment, the second bus 4091 may be a low pin count (LPC) bus. Various devices may couple to the second bus 4091 including, for example, a keyboard 4082, a mouse 4084, communication devices 4086 and a data storage unit 4088 that may store code such as the simulator logic circuitry 4096. Furthermore, an audio I/O 4090 may couple to second bus 4091. Many of the I/O devices 4092, communication devices 4086, and the data storage unit 4088 may reside on the motherboard 4005 while the keyboard 4082 and the mouse 4084 may be add-on peripherals. In other embodiments, some or all the I/O devices 4092, communication devices 4086, and the data storage unit 4088 are add-on peripherals and do not reside on the motherboard 4005.

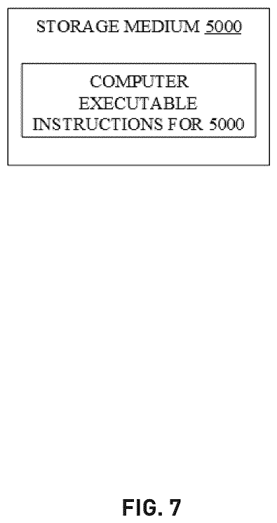

FIG. 7 illustrates an example of a storage medium 5000 to store code for simulator logic circuitry such as the simulator logic circuitry 4096 in the data storage 4088 shown in FIG. 6. Storage medium 5000 may comprise an article of manufacture. In some examples, storage medium 5000 may include any non-transitory computer readable medium or machine readable medium, such as an optical, magnetic or semiconductor storage. Storage medium 5000 may store various types of computer executable instructions, such as instructions to implement logic flows and/or techniques described herein. Examples of a computer readable or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions may include any suitable type of code, such as source code, compiled code, interpreted code, executable code, static code, dynamic code, object-oriented code, visual code, and the like. The examples are not limited in this context.

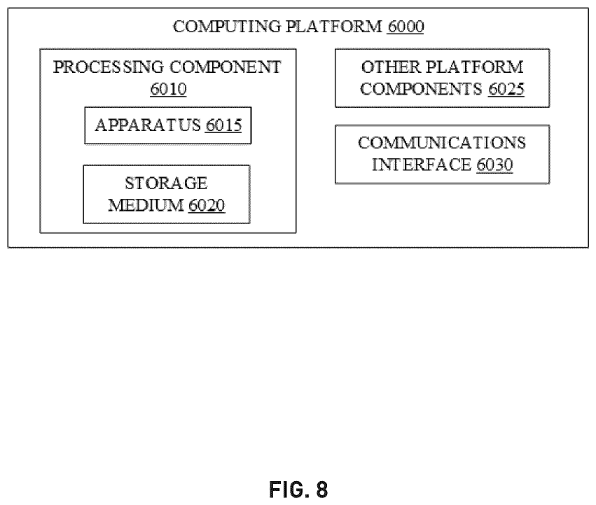

FIG. 8 illustrates an example computing platform 6000 such as the system 4000 shown in FIG. 6. In some examples, as shown in FIG. 8, computing platform 6000 may include a processing component 6010, other platform components or a communications interface 6030. According to some examples, computing platform 6000 may be implemented in a computing device such as a server in a system such as a data center or server farm that supports a manager or controller for managing configurable computing resources as mentioned above. Furthermore, the communications interface 6030 may comprise a wake-up radio (WUR) and may be capable of waking up a main radio of the computing platform 6000.

According to some examples, processing component 6010 may execute processing operations or logic for apparatus 6015 described herein such as the simulator logic circuitry 1015 and 1110 illustrated in FIG. 2A and FIG. 2B, respectively. Processing component 6010 may include various hardware elements, software elements, or a combination of both. Examples of hardware elements may include devices, logic devices, components, processors, microprocessors, circuits, processor circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), memory units, logic gates, registers, semiconductor device, chips, microchips, chip sets, and so forth. Examples of software elements, which may reside in the storage medium 6020, may include software components, programs, applications, computer programs, application programs, device drivers, system programs, software development programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an example is implemented using hardware elements and/or software elements may vary in accordance with any number of factors, such as desired computational rate, power levels, heat tolerances, processing cycle budget, input data rates, output data rates, memory resources, data bus speeds and other design or performance constraints, as desired for a given example.

In some examples, other platform components 6025 may include common computing elements, such as one or more processors, multi-core processors, co-processors, memory units, chipsets, controllers, peripherals, interfaces, oscillators, timing devices, video cards, audio cards, multimedia input/output (I/O) components (e.g., digital displays), power supplies, and so forth. Examples of memory units may include without limitation various types of computer readable and machine readable storage media in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory), solid state drives (SSD) and any other type of storage media suitable for storing information.

In some examples, communications interface 6030 may include logic and/or features to support a communication interface. For these examples, communications interface 6030 may include one or more communication interfaces that operate according to various communication protocols or standards to communicate over direct or network communication links. Direct communications may occur via use of communication protocols or standards described in one or more industry standards (including progenies and variants) such as those associated with the PCI Express specification. Network communications may occur via use of communication protocols or standards such as those described in one or more Ethernet standards promulgated by the Institute of Electrical and Electronics Engineers (IEEE). For example, one such Ethernet standard may include IEEE 802.3-2012, Carrier sense Multiple access with Collision Detection (CSMA/CD) Access Method and Physical Layer Specifications, Published in December 2012 (hereinafter “IEEE 802.3”). Network communication may also occur according to one or more OpenFlow specifications such as the OpenFlow Hardware Abstraction API Specification. Network communications may also occur according to Infiniband Architecture Specification, Volume 1, Release 1.3, published in March 2015 (“the Infiniband Architecture specification”).

Computing platform 6000 may be part of a computing device that may be, for example, a server, a server array or server farm, a web server, a network server, an Internet server, a work station, a mini-computer, a main frame computer, a supercomputer, a network appliance, a web appliance, a distributed computing system, multiprocessor systems, processor-based systems, or combination thereof. Accordingly, functions and/or specific configurations of computing platform 6000 described herein, may be included or omitted in various embodiments of computing platform 6000, as suitably desired.

The components and features of computing platform 6000 may be implemented using any combination of discrete circuitry, ASICs, logic gates and/or single chip architectures. Further, the features of computing platform 6000 may be implemented using microcontrollers, programmable logic arrays and/or microprocessors or any combination of the foregoing where suitably appropriate. It is noted that hardware, firmware and/or software elements may be collectively or individually referred to herein as “logic”.

It should be appreciated that the computing platform 6000 shown in the block diagram of FIG. 8 may represent one functionally descriptive example of many potential implementations. Accordingly, division, omission or inclusion of block functions depicted in the accompanying figures does not infer that the hardware components, circuits, software and/or elements for implementing these functions would necessarily be divided, omitted, or included in embodiments.

One or more aspects of at least one example may be implemented by representative instructions stored on at least one machine-readable medium which represents various logic within the processor, which when read by a machine, computing device or system causes the machine, computing device or system to fabricate logic to perform the techniques described herein. Such representations, known as “IP cores”, may be stored on a tangible, machine readable medium and supplied to various customers or manufacturing facilities to load into the fabrication machines that actually make the logic or processor.

Various examples may be implemented using hardware elements, software elements, or a combination of both. In some examples, hardware elements may include devices, components, processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), memory units, logic gates, registers, semiconductor device, chips, microchips, chip sets, and so forth. In some examples, software elements may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an example is implemented using hardware elements and/or software elements may vary in accordance with any number of factors, such as desired computational rate, power levels, heat tolerances, processing cycle budget, input data rates, output data rates, memory resources, data bus speeds and other design or performance constraints, as desired for a given implementation.

Some examples may include an article of manufacture or at least one computer-readable medium. A computer-readable medium may include a non-transitory storage medium to store logic. In some examples, the non-transitory storage medium may include one or more types of computer-readable storage media capable of storing electronic data, including volatile memory or non-volatile memory, removable or non-removable memory, erasable or non-erasable memory, writeable or re-writeable memory, and so forth. In some examples, the logic may include various software elements, such as software components, programs, applications, computer programs, application programs, system programs, machine programs, operating system software, middleware, firmware, software modules, routines, subroutines, functions, methods, procedures, software interfaces, API, instruction sets, computing code, computer code, code segments, computer code segments, words, values, symbols, or any combination thereof.

According to some examples, a computer-readable medium may include a non-transitory storage medium to store or maintain instructions that when executed by a machine, computing device or system, cause the machine, computing device or system to perform methods and/or operations in accordance with the described examples. The instructions may include any suitable type of code, such as source code, compiled code, interpreted code, executable code, static code, dynamic code, and the like. The instructions may be implemented according to a predefined computer language, manner or syntax, for instructing a machine, computing device or system to perform a certain function. The instructions may be implemented using any suitable high-level, low-level, object-oriented, visual, compiled and/or interpreted programming language.

Some examples may be described using the expression “in one example” or “an example” along with their derivatives. These terms mean that a particular feature, structure, or characteristic described in connection with the example is included in at least one example. The appearances of the phrase “in one example” in various places in the specification are not necessarily all referring to the same example.

Some examples may be described using the expression “coupled” and “connected” along with their derivatives. These terms are not necessarily intended as synonyms for each other. For example, descriptions using the terms “connected” and/or “coupled” may indicate that two or more elements are in direct physical or electrical contact with each other. The term “coupled,” however, may also mean that two or more elements are not in direct contact with each other, but yet still co-operate or interact with each other.

In addition, in the foregoing Detailed Description, it can be seen that various features are grouped together in a single example for the purpose of streamlining the disclosure. This method of disclosure is not to be interpreted as reflecting an intention that the claimed examples require more features than are expressly recited in each claim. Rather, as the following claims reflect, inventive subject matter lies in less than all features of a single disclosed example. Thus, the following claims are hereby incorporated into the Detailed Description, with each claim standing on its own as a separate example. In the appended claims, the terms “including” and “in which” are used as the plain-English equivalents of the respective terms “comprising” and “wherein,” respectively. Moreover, the terms “first,” “second,” “third,” and so forth, are used merely as labels, and are not intended to impose numerical requirements on their objects.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

A data processing system suitable for storing and/or executing program code will include at least one processor coupled directly or indirectly to memory elements through a system bus and/or a configuration of high-speed interconnects. The memory elements can include local memory employed during actual execution of the program code, bulk storage, and cache memories which provide temporary storage of at least some program code to reduce the number of times code must be retrieved from bulk storage during execution. The term “code” covers a broad range of software components and constructs, including applications, drivers, processes, routines, methods, modules, firmware, microcode, and subprograms. Thus, the term “code” may be used to refer to any collection of instructions which, when executed by a processing system, perform a desired operation or operations.

Logic circuitry, devices, and interfaces herein described may perform functions implemented in hardware and also implemented with code executed on one or more processors. Logic circuitry refers to the hardware or the hardware and code that implements one or more logical functions. Circuitry is hardware and may refer to one or more circuits. Each circuit may perform a particular function. A circuit of the circuitry may comprise discrete electrical components interconnected with one or more conductors, an integrated circuit, a chip package, a chip set, memory, or the like. Integrated circuits include circuits created on a substrate such as a silicon wafer and may comprise components. And integrated circuits, processor packages, chip packages, and chipsets may comprise one or more processors.

Processors may receive signals such as instructions and/or data at the input(s) and process the signals to generate the at least one output. While executing code, the code changes the physical states and characteristics of transistors that make up a processor pipeline. The physical states of the transistors translate into logical bits of ones and zeros stored in registers within the processor. The processor can transfer the physical states of the transistors into registers and transfer the physical states of the transistors to another storage medium.

A processor may comprise circuits to perform one or more sub-functions implemented to perform the overall function of the processor. One example of a processor is a state machine or an application-specific integrated circuit (ASIC) that includes at least one input and at least one output. A state machine may manipulate the at least one input to generate the at least one output by performing a predetermined series of serial and/or parallel manipulations or transformations on the at least one input.

The logic as described above may be part of the design for an integrated circuit chip. The chip design is created in a graphical computer programming language and stored in a computer storage medium or data storage medium (such as a disk, tape, physical hard drive, or virtual hard drive such as in a storage access network). If the designer does not fabricate chips or the photolithographic masks used to fabricate chips, the designer transmits the resulting design by physical means (e.g., by providing a copy of the storage medium storing the design) or electronically (e.g., through the Internet) to such entities, directly or indirectly. The stored design is then converted into the appropriate format (e.g., GDSII) for the fabrication.

The resulting integrated circuit chips can be distributed by the fabricator in raw wafer form (that is, as a single wafer that has multiple unpackaged chips), as a bare die, or in a packaged form. In the latter case, the chip is mounted in a single chip package (such as a plastic carrier, with leads that are affixed to a motherboard or other higher-level carrier) or in a multichip package (such as a ceramic carrier that has either or both surface interconnections or buried interconnections). In any case, the chip is then integrated with other chips, discrete circuit elements, and/or other signal processing devices as part of either (a) an intermediate product, such as a processor board, a server platform, or a motherboard, or (b) an end product.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority but are used to distinguish one feature from another.

In the claims, the term “comprises/comprising” does not exclude the presence of other elements, components, features, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

1. An apparatus comprising:
memory; and
logic circuitry coupled with the memory to:
interact, via one or more interface models, with a client to obtain client input to determine client data for a sequence for a type of ribonucleic acid (RNA);
analyze, via one or more intuitive models, the client data based on historical batch data and experimental batch data to identify additional client data to achieve one or more target metrics of the client data; and
simulate, by one or more process models, continuous RNA production based on the client data and the additional client data to generate one or more experimental outcomes of the continuous RNA production defined by the client data and to output a development plan for the continuous RNA production.

2. The apparatus of claim 1, wherein the one or more interface models comprise a natural language processing model, a conversational language model, a large language model, or a combination thereof, and, further comprising one or more scale-up models to scale-up the development plan to a pilot development plan or a full commercial product plan.

3. The apparatus of claim 1, the one or more process models comprising one or more machine learning models, one or more data-driven models, one or more mechanistic models, one or more hybrid models, and/or a combination thereof to simulate steps of a continuous RNA production process.

4. The apparatus of claim 3, wherein the one or more process models are based on historical batch data, peer-reviewed experimental batch data, peer-reviewed literature, or a combination thereof to simulate steps of the continuous RNA process to predict the one or more experimental outcomes.

5. The apparatus of claim 1, wherein simulation by the one or more process models identifies one or more bottlenecks in the continuous RNA production.

6. The apparatus of claim 1, the logic circuitry to further amend the client data and the additional client data to add more data or revise current data after each iteration of simulation by the process model(s) to perform additional iterations of the simulation until one or more target metrics are met.

7. The apparatus of claim 1, wherein the target metric comprises a target yield, a target purity, a target quality, a target timeline for production, a target budget, or a combination thereof.

8. The apparatus of claim 1, wherein the one or more intuitive models output suggestions after at least one or each iteration of simulation by the process model(s) for amendments to the client data, the additional client data, or both, based on historical batch data, peer-reviewed experimental batch data, peer-reviewed literature, or a combination thereof, to reach one or more of the target metrics.

9.-11. (canceled)

12. A non-transitory storage medium containing instructions, which when executed by a processor, cause the processor to perform operations, the operations to:
interact, via one or more interface models, with a client to obtain client input to determine client data for a sequence for a type of ribonucleic acid (RNA);
analyze, via one or more intuitive models, the client data based on historical batch data and experimental batch data to identify additional client data to achieve one or more target metrics of the client data; and
simulate, by one or more process models, continuous RNA production based on the client data and the additional client data to generate one or more experimental outcomes of the continuous RNA production defined by the client data and to output a development plan for the continuous RNA production.

13. The non-transitory storage medium of claim 12, wherein the one or more interface models comprise a natural language processing model, a conversational language model, a large language model, or a combination thereof.

14. The non-transitory storage medium of claim 12, the one or more process models comprising one or more machine learning models, one or more data-driven models, one or more mechanistic models, one or more hybrid models, and/or a combination thereof to simulate steps of a continuous RNA production process.

15. The non-transitory storage medium of claim 14, wherein the one or more process models are based on historical batch data, peer-reviewed experimental batch data, peer-reviewed literature, or a combination thereof to simulate steps of the continuous RNA process to predict the one or more experimental outcomes, and wherein the target metric comprises a target yield, a target purity, a target quality, a target timeline for production, a target budget, or a combination thereof.

16. The non-transitory storage medium of claim 12, wherein simulation by the one or more process models identifies one or more bottlenecks in the continuous RNA production, and further comprising one or more scale-up models to scale-up the development plan to pilot development or full commercial product.

17. The non-transitory storage medium of claim 12, the operations to further amend the client data and the additional client data to add more data or revise current data after each iteration of simulation to perform additional iterations of the simulation until one or more target metrics are met, and wherein the one or more intuitive models output suggestions for amendments to the client data, the additional client data, or both, based on historical batch data, peer-reviewed experimental batch data, peer-reviewed literature, or a combination thereof, to reach one or more of the target metrics.

18.-22. (canceled)

23. A system comprising:
data storage comprising a historical dataset, a literature dataset, and a model library; and
one or more servers comprising processors coupled with the data storage to:
interact, via one or more interface models of the model library, with a client to obtain client input to determine client data for a sequence for a type of ribonucleic acid (RNA);
analyze, via one or more intuitive models of the model library, the client data based on historical batch data of the historical dataset and experimental batch data of the literature dataset to identify additional client data to achieve one or more target metrics of the client data;
store the client data and the additional data in a client dataset of the data storage; and
simulate, by one or more process models of the model library, continuous RNA production based on the client data and the additional client data to generate one or more experimental outcomes of the continuous RNA production defined by the client data and to output a development plan for the continuous RNA production.

24. The system of claim 23, wherein the one or more interface models comprise a natural language processing model, a conversational language model, a large language model, or a combination thereof; the one or more process models comprising one or more machine learning models, one or more data-driven models, one or more mechanistic models, one or more hybrid models, and/or a combination thereof to simulate steps of a continuous RNA production process; and wherein the one or more process models are based on historical batch data, peer-reviewed experimental batch data, peer-reviewed literature, or a combination thereof to simulate steps of the continuous RNA process to predict the one or more experimental outcomes.

25.-27 (canceled)

28. The system of claim 23, the one or more servers to further amend the client data and the additional client data to add more data or revise current data after each iteration of simulation to perform additional iterations of the simulation until one or more target metrics are met, wherein the target metric comprises a target yield, a target purity, a target quality, a target timeline for production, a target budget, or a combination thereof, wherein the one or more intuitive models output suggestions for amendments to the client data, the additional client data, or both, based on historical batch data, peer-reviewed experimental batch data, peer-reviewed literature, or a combination thereof, to reach one or more of the target metrics.

29.-31. (canceled)

32. The system of claim 23, wherein simulation of continuous RNA production involves simulation of an in vitro transcription reaction, wherein the client input includes a concentration value for one or more of a DNA template, a nucleotide triphosphate, magnesium chloride and an RNA polymerase, wherein the nucleotide triphosphate is one or more of guanosine-5′-triphosphate, adenosine triphosphate, cytidine triphosphate, uridine triphosphate, pseudouridine triphosphate, dihydrouridine triphosphate, 4-thiouridine, inosine triphosphate, 7-methylguanosine triphosphate, 2,7-dimethylguanosine triphosphate, and/or 2,2,7-trimethylguanosine triphosphate.

33.-34. (canceled)

35. The system of claim 32, wherein simulation of continuous RNA production involves analyzing the client input with a pre-trained partial least squares model to obtain a predicted value of mRNA yield based on the client input; iteratively simulating the continuous RNA production to generate the one or more experimental outcomes and analyzing the client input with a pre-trained intuitive model, based on the one or more experimental outcomes, to predict changes to the client input to improve performance of the continuous RNA production with respect to a user-defined target metric; wherein the user-defined target metric includes a target yield, a target purity, a target quality, a target timeline for production, a target budget, or a combination thereof.

36. (canceled)

37. The system of claim 32, wherein simulation of continuous RNA production further includes iteratively simulating the continuous RNA production to generate the one or more experimental outcomes, analyzing the client input with a pre-trained intuitive model, based on the one or more experimental outcomes, to suggest changes to the client input to improve performance of the continuous RNA production with respect to a user-defined target metric, and interacting with the client to obtain changes to the client input after provision of the changes suggested by the pre-trained intuitive model, wherein the user-defined target metric includes a target yield, a target purity, a target quality, a target timeline for production, a target budget, or a combination thereof.
